(19) United States
(12) Patent Application Publication
(10) Pub. No.: US 2012/0052122 A1
(43) Pub. Date: Mar. 1, 2012

(54) Treatment Of Chronic Obstructive Pulmonary Disease With Phosphodiesterase-4 Inhibitor

(75) Inventor: Dirk Bredenbroeker, Konstanz (DE)

(73) Assignee: Nycomed GmbH, Konstanz (DE)

(21) Appl. No.: 13/216,936

(22) Filed: Aug. 24, 2011

Related U.S. Application Data
(60) Provisional application No. 61/377,414, filed on Aug. 26, 2010.

(51) Int. Cl. A61K 9/28; A61P 11/08; A61P 11/00; A61K 31/44; A61K 31/537

(52) U.S. Cl. 424/474; 514/352; 514/230.5

(57) ABSTRACT

Disclosed is a method of treatment of chronic obstructive pulmonary disease associated with chronic bronchitis in a patient at risk of exacerbations. The method includes administering to a patient with a history of chronic obstructive pulmonary disease associated with chronic bronchitis and at risk of exacerbations, a maintenance dose of 500 micrograms per day of roflumilast. Also disclosed is a method of increasing pre-bronchodilator FEV₁ or post-bronchodilator FEV₁ in such a patient, and increasing pre-bronchodilator FVC or post-bronchodilator FVC in such a patient. Further disclosed is a method of reducing the rate of exacerbations in such a patient.

### FIELD OF THE INVENTION

The present invention relates to the treatment of chronic obstructive pulmonary disease. More particularly, the present invention relates to the treatment of chronic obstructive pulmonary disease associated with chronic bronchitis in a human patient at risk of exacerbations, by administration of roflumilast (N-(3,5-dichloropyridin-4-yl)-3-cyclopropylmethoxy-4-difluoromethoxy-benzamide).

### BACKGROUND OF THE INVENTION

Chronic obstructive pulmonary disease (COPD) is a highly prevalent condition and a major cause of morbidity and mortality worldwide. As the disease progresses, patients with COPD may become prone to frequent exacerbations, resulting in patient anxiety, (Reference No. 1), worsening health status, lung function decline, and increase in mortality rate (References 2-4). These episodes of worsening respiratory function lead to increases in health care utilization, hospital admissions and costs. Worse, frequent exacerbations are associated with a faster decline in lung function, thereby shortening life expectancy.

Effective management of COPD involves pharmacological and non-pharmacological treatments (Reference 5). Long-acting inhaled bronchodilator drugs (β2 agonists and anticholinergic drugs) can improve health status and reduce the frequency of exacerbations, effects that are greater when long-acting β2 agonists are used in combination with inhaled corticosteroids (References 6-9). However, there is a need for further improvement of COPD therapy.

Phosphodiesterase-4 (PDE4) inhibition provides a novel approach to the treatment of COPD. Drugs that inhibit PDE4 have a wide range of anti-inflammatory actions in vitro and in vivo (References 10-12). Roflumilast, a new PDE4 inhibitor, reduces airway inflammation in COPD, as assessed with sputum neutrophil and eosinophil counts (Reference 13). However, although roflumilast improved lung function, it did not significantly reduce the frequency of exacerbations in unselected patients with severe COPD (Reference 14). The results of a post-hoc analysis of this study suggested that roflumilast reduced the rate of exacerbations in patients with severe airflow obstruction, frequent exacerbations, and those requiring oral steroids (Reference 13).

The following U.S. patents and published U.S. patent applications are hereby incorporated by reference: U.S. Pat. No. 5,712,298; U.S. Pat. No. 7,470,791; U.S. Pat. No. 7,951,397; U.S. D580,547; US2009-0171096-A1; US2006-0269600-A1; US2011-0060016. U.S. application Ser. No. 13/008,842 is also incorporated by reference. In the event of inconsistency of terminology, the present disclosure controls.

Also incorporated by reference are Calverley, P, Rabe K, et. al, Roflumilast in Symptomatic Chronic Obstructive Pulmonary Disease: Two Randomized Clinical Trials. The Lancet 2009; 374: 685-694 and Fabbri, L, Calverley, P, et. al, Roflumilast in Moderate to Severe Chronic Obstructive Pulmonary Disease Treated with Longacting Bronchodilators: Two Randomized Clinical Trials. The Lancet 2009; 374: 695-703.

### BRIEF DESCRIPTION OF THE FIGURES

FIGS. 1A and 1B are flow diagrams outlining the clinical trial profiles for the M2-124 and M2-125 studies, respectively.

FIGS. 2A and 2B are graphs depicting the probability of treatment discontinuation in the roflumilast and placebo groups for the M2-124 and M2-125 studies, respectively.

FIGS. 3A, 3B, 3C and 3D are graphs depicting prebronchodilator and postbronchodilator FEV₁ values over 52 weeks for M2-124 and M2-125 studies, respectively, (FIGS. 3A and 3B), and changes in prebronchodilator and postbronchodilator FEV₁ values over 52 weeks for M2-124 and M2-125 studies, respectively, (FIGS. 3C and 3D).

FIGS. 4A and 4B are flow diagrams outlining the clinical trial profiles for the M2-127 and M2-128 studies, respectively.

FIGS. 5A and 5B are graphs depicting the probability of treatment discontinuation in the salmeterol plus roflumilast M2-127 trial, and tiotropium plus roflumilast M2-128 trial, respectively.

FIGS. 6A and 6B are graphs depicting mean prebronchodilator and postbronchodilator FEV₁ values over 24 weeks for salmeterol plus roflumilast M2-127 trial, and tiotropium plus roflumilast M2-128 trial, respectively.

### SUMMARY OF THE INVENTION

The present invention is directed to a method of treatment of chronic obstructive pulmonary disease associated with chronic bronchitis in a patient at risk of exacerbations. The method includes administering to a patient with a history of chronic obstructive pulmonary disease associated with chronic bronchitis and at risk of exacerbations, a maintenance dose of 500 micrograms per day of roflumilast. In addition, the present invention is directed to a method of increasing pre-bronchodilator FEV₁ or post-bronchodilator FEV₁ in such a patient, and increasing pre-bronchodilator FVC or post-bronchodilator FVC in such a patient. Further, the present invention is directed to a method of reducing the rate of exacerbations in such a patient.

### DETAILED DESCRIPTION OF THE INVENTION

COPD is a highly heterogeneous disease. Current pathogenetic theories are based on complex interactions of many (incompletely defined) genetic factors that interact with many environmental factors, though the most common factor is cigarette smoking. Because of the heterogeneous nature of COPD, and the trends toward efficacy observed for roflumilast in reducing exacerbations in a broadly defined COPD population, the possibility that a subset of the COPD population might be more responsive to roflumilast-induced reduction in exacerbations was entertained.

To determine whether PDE4 inhibitors can have any effect on clinical outcomes in COPD, the hypothesis that roflumilast reduces the rate of exacerbations requiring systemic corticosteroids in specific subsets of patients with COPD was tested.

### Methods

Study M2-124 was done in 246 centers in ten countries, and study M2-125 was done in 221 centers in eight countries.

For both studies, participants were recruited from an outpatient setting if they met inclusion criteria—i.e., were former smokers or current smokers with at least a 20 pack-year history, older than 40 years, and had a clinical diagnosis of COPD (confirmed with a postbronchodilator [albuterol 400 μg] forced expiratory volume in 1 s [FEV₁]/forced vital capacity [FVC] ratio≦70%) and chronic cough and sputum production. Their postbronchodilator FEV₁ was 50% or less than the predicted value.

Predicted values for FEV₁, FVC and FEV₁/FVC, as used in the M2-124 and M2-125 studies, were calculated according to the formula of Crapo et al. and set forth below. Crapo R O, Morris A H, Gardner R M. Reference Spirometric Values Using Techniques and Equipment that Meets ATS Recommendation. Am Rev Respir Dis 1981; 123: 659-664; Knudson R J, Slatin R C, Lebowitz M D, Burrows B. The Maximal Expiratory Flow-volume Curve. Normal Standards, Variability, and Effects of Age. Am Rev Respir Dis 1976; 113(5):587-600; Knudson R J, Lebowitz M D, Holberg C J, Burrows B. Changes in the Normal Maximal Expiratory Flow-volume Curve with Growth and Aging. Am Rev Respir Dis 1983; 127(6):725-34. For African-American patients, FEV₁ and FVC values were obtained by multiplying the values obtained above by a factor of 0.88. American Thoracic Society (1991): Lung Function Testing: Selection of Reference Values and Interpretative Strategies. American Review of Respiratory Disease, Volume 144: 1202-1218.

| | | |
|---|---|---|
| FEV₁ | male | (0.0414 × height [cm]) − (0.0244 × age [y]) − 2.190 |
| | female | (0.0342 × height [cm]) − (0.0255 × age [y]) − 1.578 |
| FVC | male | (0.0600 × height [cm]) − (0.0214 × age [y]) − 4.650 |
| | female | (0.0491 × height [cm]) − (0.0216 × age [y]) − 3.590 |
| FEV₁/ | male | (−0.1300 × height [cm] − 0.152 × age [y]) + 110.49 |
| FVC | female | (−0.2020 × height [cm]) − 0.252 × age [y]) + 126.58 |

All patients had at least one recorded COPD exacerbation requiring systemic glucocorticosteroids or treatment in hospital, or both, in the previous year.

Exclusion criteria are shown below: use of theophylline was not allowed from the start of the run-in period.

COPD exacerbation (indicated by corticosteroid treatment or hospitalization) unresolved at initial visit (4 weeks pre-baseline);

Asthma or other relevant lung disease;

Clinically significant cardiopulmonary abnormalities not related to COPD;

Clinically relevant abnormal laboratory findings;

Pregnant or planned pregnancy or breast feeding;

Females of child-bearing potential not using or not willing to use a medically reliable method of contraception;

Chronic gastrointestinal (GI) disorders with a history of recurrent GI bleeds within the previous 1 year;

Participation in another clinical trial within 30 days of the run-in period;

Current participation in or completion within 3 months of the run-in period of a pulmonary rehabilitation program;

Use of disallowed drugs;

Use of immunosuppressive medications within 4 weeks prior to baseline;

Known alpha-1-antitrypsin deficiency;

Known HIV infection and/or active hepatitis;

Diagnosis or history of any cancer (other than basal cell carcinoma) within 5 years of study start;

Clinically relevant ECG findings not related to COPD and requiring further evaluation;

Alcohol or drug abuse;

Suspected hypersensitivity or contraindication to study medication;

Unable to follow study procedures (e.g. language difficulties, psychological disorders); and

Suspected non-compliance.

The studies were approved by local ethical review committees and done in accordance with the Declaration of Helsinki and Good Clinical Practice guidelines. All patients provided written informed consent.

Each trial had an initial 4-week run-in, during which patients took a placebo tablet once a day in the morning, and recorded their use of short-acting bronchodilator drugs, and production of cough and sputum on their daily diary cards.

Cough and sputum production scores were tallied as follows. The patient diary filled out by each patient on a daily basis, included a chart for the patient to self-assess cough and sputum production. The patient was requested to “assess your symptoms of cough and sputum production for the last 24 hours.” For cough, the patient was asked: “How was your cough today?” and asked to rank the cough as follows: 0 for no cough; 1 for mild cough (at some time during the day); 2 for moderate cough (regularly during the day); and 3 for severe cough (never free of cough or feeling free of the need to cough). For sputum, the patient was asked: “How much inconvenience was caused by your sputum today?” and asked to rank the sputum production as follows: 0 for none (unnoticeable); 1 for mild (noticeable as a problem); 2 moderate (frequent inconvenience); 3 severe (constant problem).

In this initial study phase, patients, but not investigators, were unaware of the treatment they were assigned to. Patients were then randomly assigned to oral roflumilast 500 μg administered as a tablet once a day or placebo, taken in the morning for the subsequent 52 weeks, provided that the total of their cough and sputum scores was greater than 14 in the week before randomization, the haemoccult (guaiac) test during the baseline period was negative, at least 80% of prescribed placebo tablets were taken, and patients were clinically stable. Patients could use short acting β2 agonists as needed and could continue treatment with long acting β2 agonists or short acting anti-cholinergic drugs at stable doses. However, inhaled corticosteroids and long acting anti-cholinergic drugs were not allowed during the study. Eligible patients were stratified according to their use of long acting β2 agonists and smoking status.

### Randomization and Masking

A randomization list of patient random numbers was generated using a pseudorandom number generator. In the double-blind treatment phase, all individuals involved in the studies were unaware of treatment assignment—tablets were identical in appearance. The investigator or anyone at the study site was prevented from knowing the allocation sequence with code labeling. The sponsor and clinical research associate were notified if there was a clinical reason for an individual's treatment to be unmasked by the investigator with the interactive voice recognition system.

After randomization, patients were assessed every 4 weeks up to week 12 and every 8 weeks thereafter. At each visit, spirometric measurements were recorded before and 15-45 min after administration of bronchodilator (inhaled albuterol 400 μg). Additionally, any new exacerbations or adverse events were recorded, as were the patient's bodyweight, adherence to tablets, completeness of the daily diary records, use of short acting β2 agonists, and investigator-administered transition dyspnea index (TDI), (Reference 15) and dispensed study medication.

### Study Endpoints

The primary endpoints were the change in prebronchodilator FEV₁ during treatment and the rate of COPD exacerbations, defined as moderate if they required treatment with oral or parenteral corticosteroids, or severe if they were associated with hospital admission or death. Key secondary outcomes included the postbronchodilator FEV₁ (change from baseline during treatment), time to death from any cause, natural log-transformed C-reactive protein concentration (a possible marker of systemic inflammation in COPD; (Reference 16) change from baseline to study end) and TDI focal score (during treatment). A change of one unit in the TDI focal score was considered clinically significant. Additionally, data for the total number of COPD exacerbations (as defined above together with episodes treated with antibiotics alone) and a range of spirometric outcomes were gathered. As part of a planned health economic analysis (data for subsequent presentation), patients completed the Euroquol 5-dimension (EQ-5D) questionnaire, a measure of health utility, at each visit (Reference 17).

Bodyweight was measured with the same scales at each visit, height was measured with a stadiometer, and body-mass index (BMI) was calculated. At weeks 28 and 52 after randomization, blood samples were taken for routine haematology and biochemistry tests, and an electrocardiogram (ECG) was done. In study M2-125, 24-h Holter monitoring was undertaken at 19 sites to identify any arrhythmias.

### Statistical Analysis

With the exception of the post-hoc investigation of adverse events and bodyweight, all reported efficacy analyses were pre-specified in the intention-to-treat population. Data are presented as mean and SD, unless otherwise indicated. On the basis of an assumption of a mean exacerbation rate of 1.25 per patient per year in the placebo group and 1.00 in the roflumilast group, and using a Poisson regression model, with a correction for over dispersion of 2 based on previous data, (Reference 1.4) it was estimated that 750 patients per treatment group in each trial would provide 90% power to detect a significant difference between treatments with a two-sided α level of 0.05.

A negative binominal regression analysis was done to assess the robustness of the results against the distributional assumptions. Data were analyzed in the two studies separately and in a pooled analysis. Changes were analyzed from baseline in prebronchodilator and postbronchodilator FEV₁ using a repeated-measures analysis of covariance with all data available for patients during the 52-week treatment. (Reference 18) A Cox proportional hazard model was used to test for differences in time-to-event data. For analysis of the concentrations of C-reactive protein, an analysis of covariance model was used, with the method of the last observation carried forward for the log-transformed data for concentrations. For the regression models (analysis of covariance, Cox, and Poisson), the covariates included treatment, age, sex, smoking status (current or former smoker), country, and treatment with long acting β2 agonists. In the Cox analysis, country was included as a stratum. In the Poisson regression analysis, baseline postbronchodilator FEV₁ (% of predicted value) was also included as a covariate. To address the issue of multiple comparisons, a hierarchical hypothesis-testing approach was adopted, If the primary outcomes were positive, the key secondary outcomes were tested in the order above. If a significant difference between treatments was not obtained for the primary or key secondary outcomes, all subsequent analyses were considered exploratory. No interim analyses were done in either study before unmasking. However, several statistical analyses were preplanned and done to assess the robustness of the results with respect to the effect of differential dropouts and missing data. Adverse events were analyzed with descriptive statistics and 95% CIs for the differences between treatments.

The trials are registered with ClinicalTrials.gov, number NCT00297102 for M2-124, and NCT00297115 for M2-125.

### Results

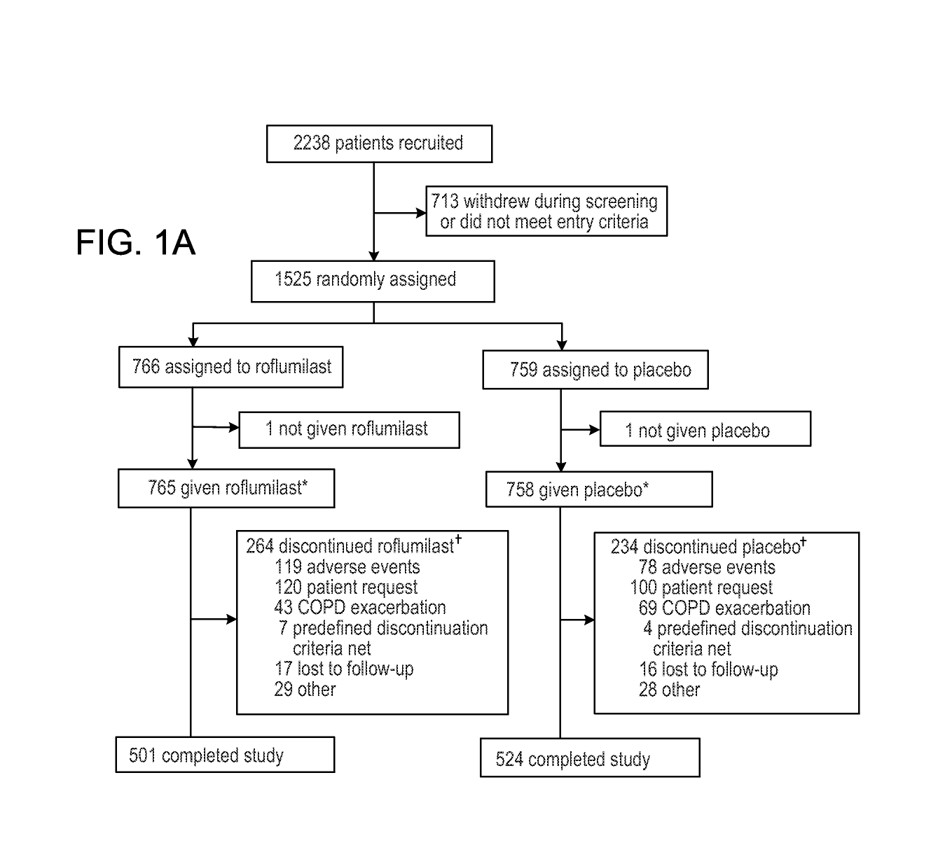

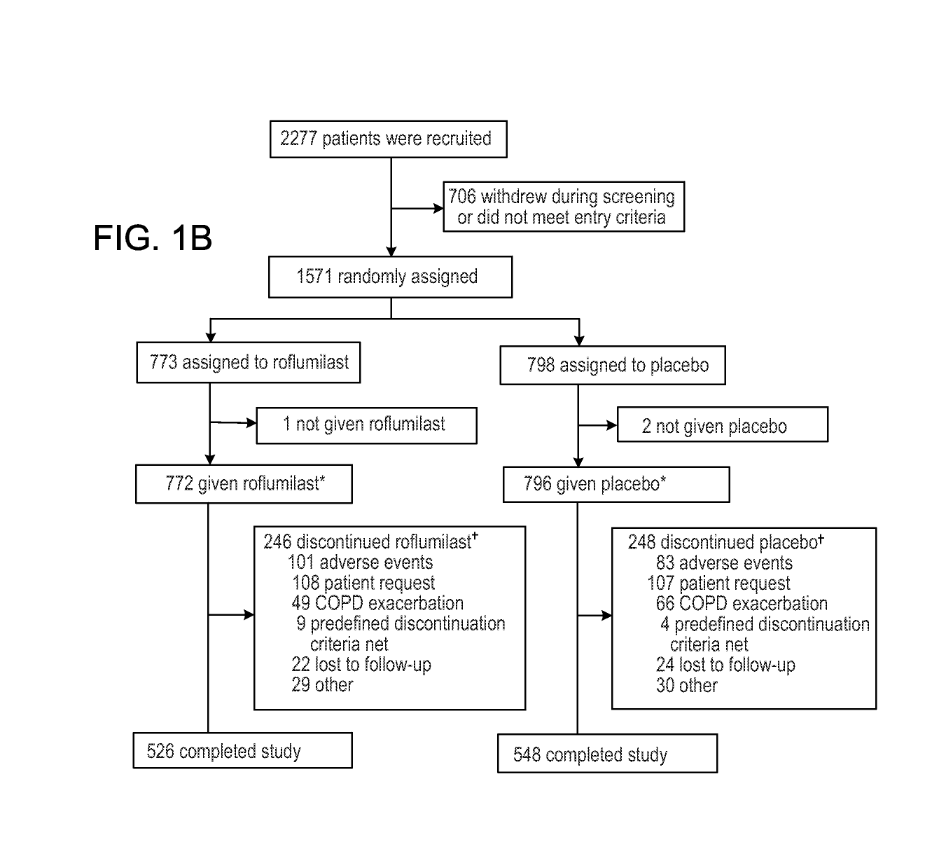

In the M2-124 study, 1523 patients were randomly assigned and treated (FIG. 1A). In M2-125, 1568 patients were randomly assigned and treated (FIG. 1B). Four patients in M2-124 and six in M2-125 were given roflumilast rather than placebo and are included in the treated group for the safety analysis. Table 1 shows the demographic and baseline characteristics of the patients who took at least one dose of study medication. The only difference between the trials was the proportion of Asian patients.

TABLE 1
Demographics and baseline characteristics of the intention-to-treat populations in the M2-124 and M2-125 trials.

| | M2-124 | | M2-125 | | M2-124 and M2-125 | |
|---|---|---|---|---|---|---|
| | Roflumilast | Placebo | Roflumilast | Placebo | Roflumilast | |
| | | | | | | Placebo |
| Age (years)* | 64 (10) | 63 (9) | 64 (9) | 64 (9) | 64 (9) | 64 (9) |
| Men | 540 (71%) | 538 (71%) | 610 (79%) | 648 (81%) | 1150 (75%) | 1186 (76%) |
| Cigarette pack-year*† | 48 (24) | 46 (23) | 49 (26) | 47 (24) | 48 (25) | 47 (23) |
| Smoking Status* | | | | | | |
| Current Smoker | 365 (48%) | 361 (48%) | 270 (35%) | 282 (35%) | 635 (41%) | 643 (41%) |
| Former Smoker | 400 (52%) | 397 (52%) | 502 (65%) | 514 (65%) | 902 (59%) | 911 (59%) |
| Prebronchodillator FEV₁(L)‡ | 1.07 (0.4) | 1.06 (0.4) | 0.95 (0.3) | 0.98 (0.4) | 1.01 (0.4) | 1.02 (0.4) |
| Postbronchodillator FEV₁(L)‡ | 1.16 (0.4) | 1.15 (0.4) | 1.05 (0.4) | 1.07 (0.4) | 1.10 (0.4) | 1.11 (0.4) |
| Prebronchodillartor FEV₁ (% of | 34.7 (10.2) | 34.6 (10.3) | 31.4 (10.1) | 32.2 (10.8) | 33.0 (10.3) | 33.4 (10.6) |
| predicted)‡ | | | | | | |
| Postbronchodillator FEV₁ (% of | 37.6 (10.7) | 37.5 (10.4) | 34.6 (10.3) | 35.3 (10.9) | 36.1 (10.6) | 36.4 (10.7) |
| predicted)‡ | | | | | | |
| Postbronchodillator FEV₁/FVC (%)‡ | 43.3 (11.6) | 42.7 (11.0) | 41.2 (10.7) | 41.3 (10.8) | 42.3 (11.2) | 42.0 (10.9) |
| COPD severity*∫ | | | | | | |
| Severe | 486 (64%) | 510 (67%) | 457 (59%) | 479 (60%) | 943 (61%) | 989 (64%) |
| Very severe | 199 (26%) | 184 (24%) | 264 (34%) | 256 (32%) | 463 (30%) | 440 (28%) |
| Body mass index (kg/m²)‡ | 26.4 (5.5) | 26.0 (5.5) | 25.2 (6.2) | 25.4 (5.9) | 25.8 (5.9) | 25.7 (5.7) |
| C-reactive protein (mg/L) | 8.1 (14.0) | 7.2 (12.5) | 8.3 (14.6) | 9.2 (17.6) | 8.2 (14.3) | 8.2 (15.4) |
| Concomitant treatment with | 378 (49%) | 385 (51%) | 371 (48%) | 408 (51%) | 749 (49%) | 793 (51%) |
| longacting β₂ agonists\|\| | | | | | | |
| | | | | | | Placebo |
| | | | | | | 537 (35%) |
| Concomitant treatment with | 240 (31%) | 245 (32%) | 297 (38%) | 324 (41%) | 537 (35%) | 537 (35%) |
| shortacting nticholinergics | | | | | | |
| Concomitant treatment with | 761 (99%) | 753 (99%) | 769 (100%) | 791 (99%) | 1530 (100%) | 1544 (99%) |
| longacting β₂ agonists\|\| | | | | | | |
| Pretreatment with inhaled | 338 (44%) | 335 (44%) | 312 (40%) | 322 (40%) | 650 (42%) | 657 (42%) |
| corticosteroids** | | | | | | |
| Ethnic origin | | | | | | |
| Asian | 1 (<1%) | 1 (<1%) | 174 (23%) | 179 (22%) | 175 (11%) | 180 (12%) |
| Native American | 0 | 1 (<1%) | 2 (<1%) | 1 (<1%) | 2 (<1%) | 2 (<1%) |
| Black | 11 (1%) | 15 (2%) | 8 (1%) | 14 (2%) | 19 (1%) | 29 (2%) |
| White | 737 (96%) | 732 (97%) | 559 (72%) | 568 (71%) | 1296 (84%) | 1300 (84%) |
| Other | 16 (2%) | 9 (1%) | 29 (4%) | 34 (4%) | 45 (3%) | 43 (3%) |

Data are number (%) or mean (SD).
FEV₁ = forced expiratory volume in 1s.
FVC = forced vital capacity.
COPD = chronic obstructive pulmonary disease.
*Measurements were taken at the beginning of the run-in period.
†1 pack-year = 20 cigarettes per day for 1 year.
‡Measurements were taken at baseline.
∫Based on the criteria of the Global Initiative for chronic Obstructive Lung Disease.
¶Percentages do not add up to 100% because patients with mild COPD are not shown.
||Based on whether the patient had used medications at least once within the start and up to the end of the treatment period inclusive.
**Based on whether the patient had used inhaled corticosteroids at least once within the period starting the day after the first visit until the day before randomization, inclusive.

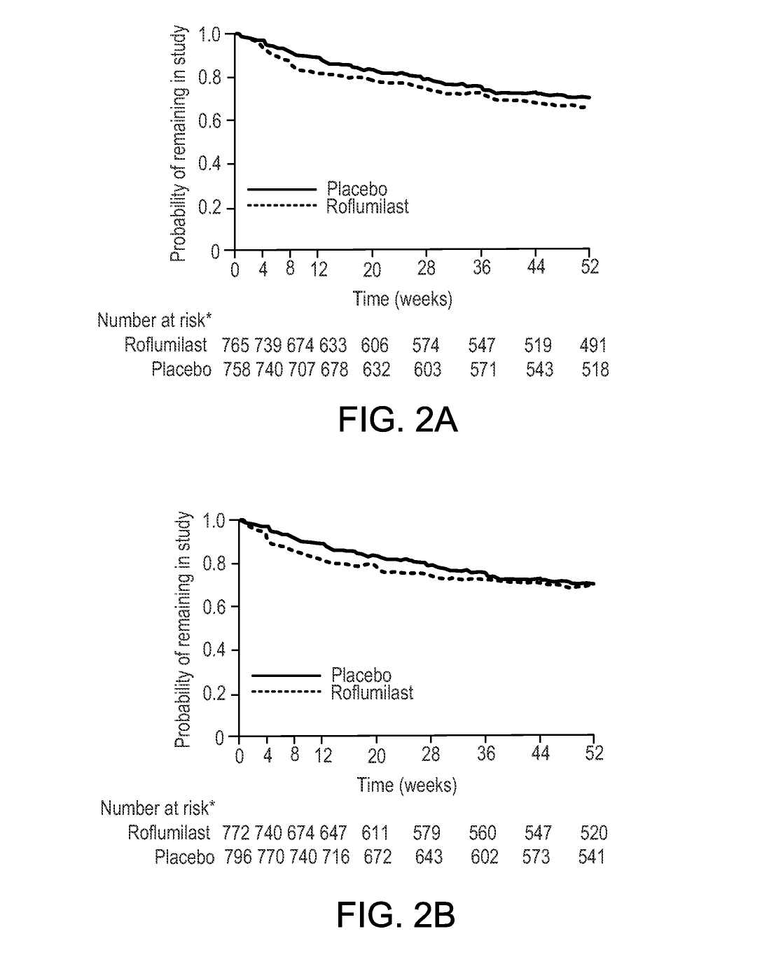

The mean prebronchodilator FEV₁ was between 31% and 35% of predicted value in the different study subgroups; 40-44% had used inhaled corticosteroids previously, whereas about 50% used long acting β2 agonists during the trials (Table 1). Patient withdrawal was similar in the roflumilast and placebo groups (35% and 31%, respectively; in M2-124, and 32% and 31%, respectively, in M2-125; FIG. 1), However, more patients in the roflumilast group than in the placebo group withdrew in the first 12 weeks after randomization (FIGS. 2A and 2B).

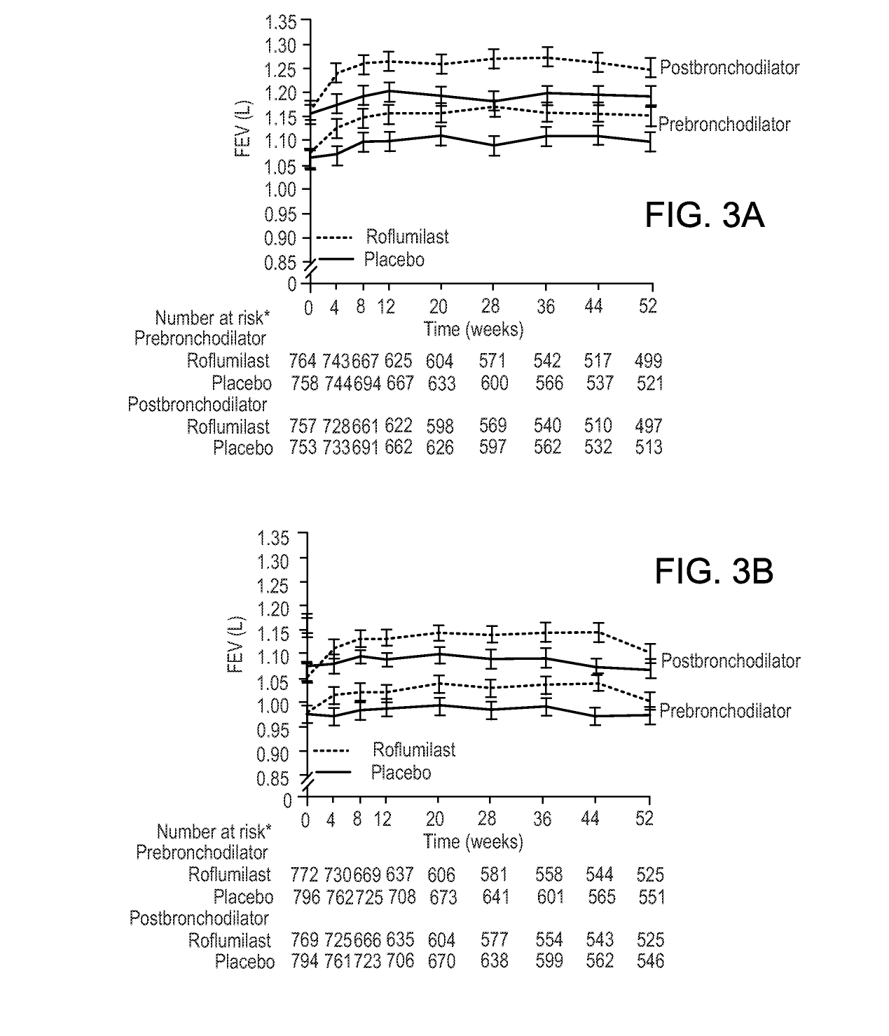

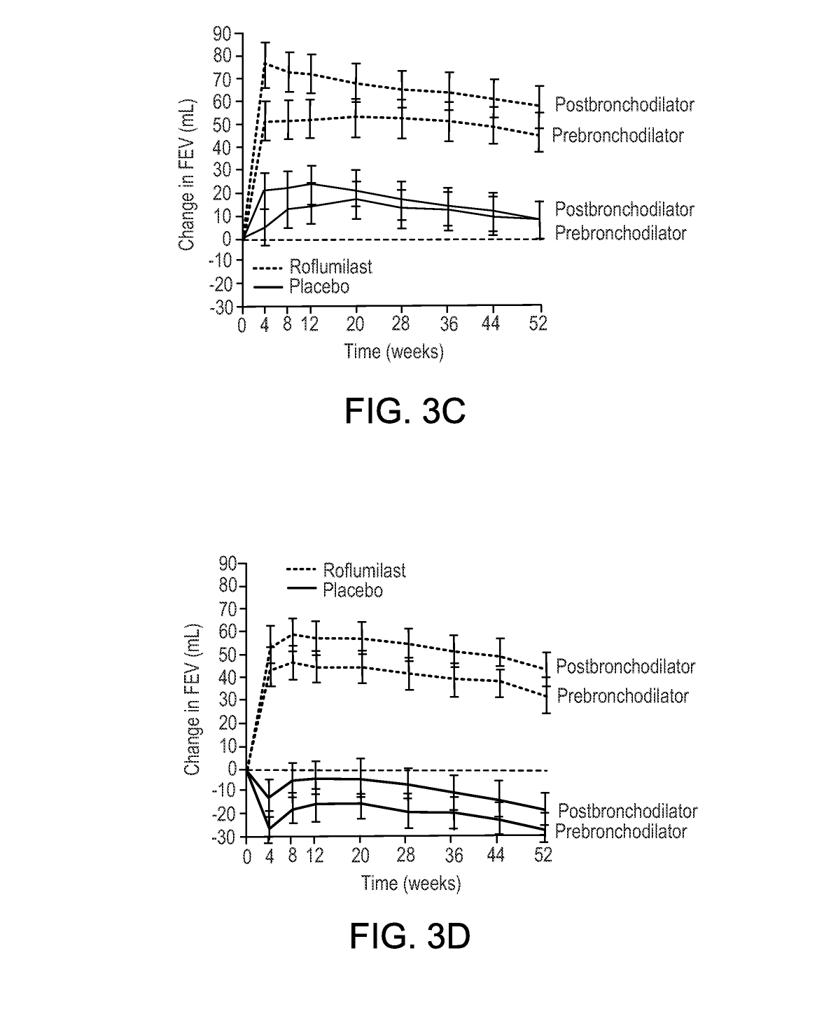

Adherence to treatment was similar in all groups: mean compliance was 93% (SD 25) in the roflumilast group and 95% (Reference 14) in the placebo group in the M2-124 study, and 93% (Reference 16) in the roflumilast group and 96% (Reference 15) in the placebo group in the M2-125 study. The primary endpoints were achieved in both studies. FIGS. 3A to 3D shows the FEV₁ data during the studies; Table 2 shows the summary results. In the pooled analysis, prebronchodilator FEV₁ increased from baseline in the roflumilast group and decreased in the placebo group (Table 2).

TABLE 2
Lung function variables, exacerbations, and other clinical outcomes

| | M2-124 | | | M2-125 | | |
|---|---|---|---|---|---|---|
| | | | Roflumilast vs | | | Roflumilast vs |
| | Roflumilast | Placebo | Placebo | Roflumilast | Placebo | Placebo |
| Lung Function* | | | | | | |
| Change in | 46 (8); | 8 (8); | Difference 39 (18 | 33 (7); n = 730 | −25 (7); | Difference 58 (41 |
| prebronchodillator FEV₁ | n = 745 | n = 745 | to 60); p = 0.0003 | | n = 766 | to 75); p < 0.0001 |
| (mL) | | | | | | |
| Change in | 57 (9); | 8 (8); | Difference 49 (26 | 44 (7); n = 724 | −17 (7); | Difference 61 (44 |
| postbronchodillator | n = 729 | n = 736 | to 71); p < 0.0001 | | n = 764 | to 79); p < 0.0001 |
| FEV₁ (mL) | | | | | | |
| Change in | 68 (15); | −21 (15); | Difference 89 (51 | 60 (14); | −48 (14); | Difference 108 |
| prebronchodillator FVC | n = 745 | n = 745 | to 127); p < 0.0001 | n = 730 | n = 766 | (75 to 141); |
| (mL) | | | | | | p < 0.0001 |
| Change in | 76 (15); | −25 (15); | Difference 101 (63 | 58 (13); | −45 (13); | Difference 103 |
| postbronchodillator | n = 729 | n = 736 | to 139); p < 0.0001 | n = 724 | n = 764 | (72 to 134); |
| FVC (mL) | | | | | | p < 0.0001 |
| Change in | 0.314 | 0.001 | Difference 0.312 (−0.262 | 0.200 | 0.309 | Difference 0.510 |
| prebronchodillator | (0.223); | (0.219); | to 0.886); | (0.190); | (0.186); | (0.061 to 0.958); |
| FEV₁/FVC (%) | n = 745 | n = 745 | p = 0.2858 | n = 730 | n = 766 | p = 0.0261 |
| Change in | 0.488 | 0.286 | Difference 0.202 (−0.343 | 0.552 | −0.115 | Difference 0.668 |
| postbronchodillator | (0.211); | (0.208); | to 0.747); | (0.186); | (0.182); | (0.226 to 1.109); |
| FEV₁/FVC (%) | n = 729 | n = 736 | p = 0.4674 | n = 724 | n = 764 | p = 0.0031 |
| Change in | 19 (5); | 2 (5); | Difference 17 (3 to | 15 (5); n = 730 | −10 (5); | Difference 25 (13 |
| prebronchodillator | n = 745 | n = 745 | 30); p = 0.0152 | | n = 765 | to 36); p < 0.0001 |
| FEF25-75 (mL/s) | | | | | | |
| Change in | 22 (6); | 12 (6); | Difference 11 (−5 | 21 (5); n = 724 | −8 (5); | Difference 29 (18 |
| postbronchodillator | n = 729 | n = 736 | to −27); p = 0.1809 | | n = 763 | to 40); p < 0.0001 |
| FEF25-75 (mL/s) | | | | | | |
| Change in | 6.65 (1.45); | 3.58 | Difference 30.7 (−0.66 | 0.75 (1.45); | −3.09 | Difference 3.85 |
| prebronchodillator PEF | n = 745 | (1.43); | to 6.81); | n = 730 | (1.41); | (0.46 to 7.23); |
| (L/min) | | n = 745 | p = 0.1063 | | n = 766 | p = 0.0261 |
| Change in | 8.08 (1.50); | 3.87 | Difference 4.21 | 1.93 (1.49); | −3.14 | Difference 5.07 |
| postbronchodillator PEF | n = 729 | (1.48); | (0.34 to 8.07); | n = 724 | (1.45); | (1.60 to 8.53); |
| (L/min) | | n = 736 | p = 0.0328 | | n = 764 | p = 0.0042 |
| Exacerbations‡ | | | | | | |
| Moderate or severe | 1.08 (0.96-1.21); | 1.27 | RR 0.85 (0.74 to | 1.21 (1.07-1.36); | 1.49 | RR 0.82 (0.71 to |
| (mean rate, per patient, | n = 344 | (1.14-1.40); | 0.98); p = 0.0278) | n = 373 | (1.33-1.66); | 0.94); p = 0.0035 |
| per year [95% CI]) | | n = 389 | | | n = 432 | |
| Severe (mean rate, per | 0.11 (0.07-0.15); | 0.12 | RR 0.89 (0.61 to | 0.14 (0.10-0.20); | 0.18 | RR 0.77 (0.53 to |
| patient, per year | n = 69 | (0.09-0.16); | 1.29); p = 0.5273 | n = 88 | (0.13-0.25); | 1.11); p = 0.1656 |
| [95% CI]) | | n = 81 | | | n = 117 | |
| Moderate (mean rate, | 0.94 (0.83-1.06); | 1.11 | RR 0.84 (0.72 to | 1.04 (0.92-1.18); | 1.27 | RR 0.82 (0.71-0.95); |
| per patient, per year | n = 299 | (1.00-1.25); | 0.99); p = 0.0325 | n = 325 | (1.13-1.42); | p = 0.0075 |
| [95% CI]) | | n = 343 | | | n = 380 | |
| Treated with systemic | 1.10 (0.98-1.23); | 1.30 | RR 0.85 (0.74 to | 1.17 (1.04-1.31); | 1.41 | RR 0.83 (0.72 to |
| corticosteroids, | n = 336 | (1.17-1.43); | 0.98; p = 0.0240 | n = 364 | (1.27-1.57); | 0.95); p = 0.0055 |
| antibiotics, or both | | n = 382 | | | n = 416 | |
| (mean rate, per patient, | | | | | | |
| per year [95% CI]) | | | | | | |
| Median time to first | 85.0 (29.5-185.5) | 71.0 | HR 0.88 (0.76 to | 73.0 (26.0-195.0); | 69.5 | HR 0.89 (0.78 to |
| exacerbation (moderate | | (29.0-152.0) | 1.02); p = 0.0859 | | (27.0-169.5) | 1.03); p = 0.1132 |
| or severe, days [IQR]) | | | | | | |
| Median time to second | 172.0 | 159.0 | HR 0.79 (0.64 to | 188.0 (84.0-281.0) | 144.0 | HR 0.79 (0.65 to |
| exacerbation (moderate | (102.0-253.0) | (97.0-229.0) | 0.98); p = 0.0290 | | (81.0-239.0) | 0.97); p = 0.0214 |
| or severe, days [IQR]) | | | | | | |
| Further pre-specified | | | | | | |
| secondary analyses | | | | | | |
| TDI focal score* | 0.7 (0.1); | 0.4 (0.1); | Difference 0.2 (0.0 | 0.7 (0.1); | 0.4 (0.1); | Difference 0.3 |
| | n = 741 | n = 745 | to 0.4); p = 0.0356 | n = 729 | n = 769 | (0.1 to 0.5); |
| | | | | | | p = 0.0059 |
| Change in C-reactive | 1.0; n = 691 | 1.1; | Difference 1.0 (0.8 | 1.1; n = 680 | 1.0; | Difference 1.1 |
| protein from baseline to | | n = 694 | to 1.1); p = 0.4089 | | n = 696 | (0.9 to 1.2); |
| last postrandomisation | | | | | | p = 0.3627 |
| visit (mg/L)* | | | | | | |
| Time to mortality (days; | 213.8 | 207.5 | HR 1.0 (0.5 to 20.); | 201.0 | 214.6 | HR 1.2 (0.7 to |
| mean, SD) | (118.9); | (108.5); | p = 0.9212 | (116.9); | (137.3); | 2.1); p = 0.5028 |
| | n = 765 | n = 758 | | n = 772 | n = 796 | |
| Health utility | | | | | | |
| assessment | | | | | | |
| EQ-5D total score* | 0.0049 | 0.0097 | Difference −0.0047 | 0.0100 | −0.0006 | Difference 0.0106 |
| | (0.0058); | (0.0057); | (−0.0196 to | (0.0065); | (0.0063); | (−0.0046 to |
| | n = 743 | n = 740 | 0.0101); p = 0.5331 | n = 727 | n = 764 | 0.0257); p = 01715 |

| | M2-124 and M2-125 | | |
|---|---|---|---|
| | | | Roflumilast vs |
| | Roflumilast | Placebo | Placebo |
| Lung Function* | | | |
| Change in | 40 (6); | −9 (5); | Difference 48 |
| prebronchodillator FEV₁ | n = 1475 | n = 1511 | (35 to 62); |
| (mL) | | | p < 0.0001 |
| Change in | 50 (6); | −4 (6); | Difference 55 |
| postbronchodillator | n = 1453 | n = 1500 | (41 to 69); |
| FEV₁ (mL) | | | p < 0.0001 |
| Change in | 64 (10); | −34 (10); | Difference 98 |
| prebronchodillator FVC | n = 1475 | n = 1511 | (73 to 123); |
| (mL) | | | p < 0.0001 |
| Change in | 67 (10); | −35 (10); | Difference 101 |
| postbronchodillator | n = 1453 | n = 1500 | (77 to 126); |
| FVC (mL) | | | p < 0.0001 |
| Change in | 0.247 | −0.146 | Difference 0.393 |
| prebronchodillator | (0.147); | (0.1439); | (0.028 to 0.758); |
| FEV₁/FVC (%) | n = 1475 | n = 1511 | p = 0.0350 |
| Change in | 0.517 | 0.090 | Difference 0.426 |
| postbronchodillator | (0.141); | (0.138); | (0.077 to 0.776); |
| FEV₁/FVC (%) | n = 1453 | n = 1500 | p = 0.0169 |
| Change in | 16 (4); | −4 (4); | Difference 20 |
| prebronchodillator | n = 1475 | n = 1510 | (12 to 29); |
| FEF25-75 (mL/s) | | | p < 0.0001 |
| Change in | 21 (4); | 2 (4); | Difference 19 |
| postbronchodillator | n = 1453 | n = 1499 | (10 to 29); |
| FEF25-75 (mL/s) | | | p < 0.0001 |
| Change in | 3.69 (1.02); | 0.17 | Difference 3.53 |
| prebronchodillator PEF | n = 1475 | (0.99); | (1.01 to 6.04); |
| (L/min) | | n = 1511 | p = 0.0060 |
| Change in | 4.93 (1.05); | 0.22 | Difference 4.72 |
| postbronchodillator PEF | n = 1453 | (1.02); | (2.13 to 7.30); |
| (L/min) | | n = 1500 | p = 0.0004 |
| Exacerbations‡ | | | |
| Moderate or severe | 1.14 (1.05-1.24); | 1.37 | RR 0.83 (0.75 to |
| (mean rate, per patient, | n = 717 | (1.28-1.48); | 0.92); p = 0.0003 |
| per year [95% CI]) | | n = 821 | |
| Severe (mean rate, per | 0.12 (0.10-0.16); | 0.15 | RR 0.82 (0.63 to |
| patient, per year | n = 157 | (0.12-0.19); | 1.06); p = 0.1334 |
| [95% CI]) | | n = 198 | |
| Moderate (mean rate, | 0.99 (0.91-1.08); | 1.19 | RR 0.83 (0.75 to |
| per patient, per year | n = 624 | (1.10-1.29); | 0.92); p = 0.0007 |
| [95% CI]) | | n = 723 | |
| Treated with systemic | 1.13 (1.04-1.23); | 1.35 | RR 0.84 (0.76 to |
| corticosteroids, | n = 700 | (1.26-1.46); | 0.92); p = 0.0003 |
| antibiotics, or both | | n = 798 | |
| (mean rate, per patient, | | | |
| per year [95% CI]) | | | |
| Median time to first | 80.0 (28.0-190.0) | 71.0 | HR 0.89 (0.80 to |
| exacerbation (moderate | | (28.0-160.0) | 0.98); p = 0.0185 |
| or severe, days [IQR]) | | | |
| Median time to second | 177.0 (92.0-262.0) | 148.0 | HR 0.79 (0.69 to |
| exacerbation (moderate | | (85.0-236.0) | 0.91); p = 0.0014 |
| or severe, days [IQR]) | | | |
| Further pre-specified | | | |
| secondary analyses | | | |
| TDI focal score* | 0.7 (0.1); | 0.4 (0.1); | Difference 0.3 |
| | n = 1470 | n = 1514 | (0.1 to 0.4); |
| | | | p = 0.0009 |
| Change in C-reactive | 1.1; n = 1371 | 1.1; | Difference 1.0 |
| protein from baseline to | | n = 1390 | (0.9 to 1.1); |
| last postrandomisation | | | p = 0.8670 |
| visit (mg/L)* | | | |
| Time to mortality (days; | 206.1 | 211.7 | HR 1.1 (0.7 to |
| mean, SD) | (116.4); | (125.1); | 1.8); p = 0.5452 |
| | n = 1537 | n = 1554 | |
| Health utility | | | |
| assessment | | | |
| EQ-5D total score* | 0.0072 | 0.0049 | Difference |
| | (0.0043); | (0.0042); | 0.0023 (−0.0083 |
| | n = 1470 | n = 1504 | to 0.0129); |
| | | | p = 0.6712 |

Data are mean (SE), mean difference (95% CI), or point estimate (95% CI), unless otherwise indicated.
n = number of patients with data available (or, for exacerbations, number of patients with at least one exacerbation).
FEV₁ = forced expiratory volume in 1s.
FVC = forced vital capacity.
FEF = forced expiratory flow.
PEF = peak expiratory flow.
RR = rate ratio.
HR—hazard ratio.
TDI = transition dyspnea index.
EQ-5D = Euroquol 5-dimension,
*Least squares means (SE).
†Estimated exacerbation rates were based on a Poisson regression model and HRs were based on a Cox proportional hazards model.
‡Since patients might have had more than one type of exacerbation, the total of moderate and severe exacerbations is different from the total of exacerbations that were moderate or severe.

The postbronchodilator FEV₁, a secondary outcome variable, increased significantly from baseline with roflumilast compared with placebo in both studies and in the pooled analysis (Table 2), Prebronchodilator FVC was significantly greater with roflumilast than with placebo in both studies (Table 2). Similar significant improvements were seen in postbronchodilator PVC and prebronchodilator midexpiratory flow. These changes in lung function were similar with and without treatment with long acting β2 agonist (mean prebronchodilator FEV₁ increase with long acting β2 agonist, 46 ml. [p<0.0001] and without long acting β2 agonist, 50 mL [p<0.0001]).

In the pooled analysis, the estimated rate of exacerbations per patient per year that were moderate or severe was 17% lower in the roflumilast group than in the placebo group (Table 2). These findings were supported by the negative binomial regression analysis (data not shown). The difference in rates between treatments was independent of concomitant long acting β2 agonist use (p=0.5382, treatment by concomitant treatment with long acting β2 agonist interaction). The total number of exacerbations (excluding severe events) requiring treatment with systemic corticosteroids or antibiotics, or both, was also lower in the roflumilast group than in the placebo group (reduction 16%) in the pooled analysis (Table 2). The times to the first and second episodes of exacerbations that were moderate or severe were significantly prolonged (Table 2). When the analysis was restricted to patients who completed the trials, similar differences in exacerbation rates were seen between the groups, although these were not significant. The pre-planned sensitivity analyses confirmed the robustness of results for the primary endpoints with respect to the effect of dropouts and missing data (data not shown).

A total of 84 patients died during the studies. The mortality rates per year did not differ in the roflumilast and placebo groups in the M2-124 study (17 [2%] vs 17 [2%]), and in the roflumilast and placebo groups in the M2-125 study (25 [3%] vs 25 [3%]; hazard ratio for time to death from any cause was >1 in both studies; Table 2). Baseline concentrations of C-reactive protein varied widely and did not change significantly during the study or with treatment. A small improvement was noted in TDI focal score from baseline with roflumilast compared with placebo but there were no differences in total EQ-5D scores (Table 2). Adverse events in the pooled study population were reported by 1040 (67%) patients in the roflumilast group and 963 (62%) in the placebo group; serious adverse events were reported by 301 (19%) and 336 (22%) patients, respectively.

Discontinuations associated with adverse events were more common in the pooled roflumilast groups than in the pooled placebo groups (219 [14%] vs 177 [11%]). With the exception of COPD, the most frequent adverse events leading to discontinuation were diarrhea, nausea, and headache in the pooled analysis (data not shown). The probability of withdrawal due to adverse events in the first 12 weeks was higher in roflumilast-treated patients (8% in both studies) than in placebo-treated patients (3% in both studies). The subsequent probability of withdrawal because of adverse events was similar between treatments (9% of roflumilast-treated patients in both studies, and 9% of placebo-treated patients in both studies).

Vomiting was reported by 17 (1%) patients in the roflumilast groups and 11 (<1%) in the placebo groups. More patients in the roflumilast than in the placebo groups had weight loss (Table 3). The mean weight change was a reduction of 2.09 kg (SD 3.98) with roflumilast after 1 year and an increase of 0.08 kg (3.48) with placebo. The change in weight in the roflumilast group happened in the first 6 months of treatment and was attenuated thereafter. Patients in the roflumilast group reporting diarrhea, nausea, vomiting, or headache had greater weight loss than did those not reporting these symptoms (2.60 kg [3.72] vs 2.02 kg [4.01]). The largest absolute weight loss with roflumilast occurred in obese patients (BMI>30). No differences were noted in the proportion of reported cardiovascular adverse events in the roflumilast and placebo groups (108 [7%] and 120 [8%], respectively). Atrial fibrillation was an infrequent complication reported by 17 (1%) patients in the roflumilast groups and 7 (<1%) of those in the placebo groups. There was no difference between roflumilast and placebo groups in the occurrence of rhythm disturbances in 33 and 22 Holter-monitored recordings, respectively. The incidence of pneumonia or other pulmonary infections did not increase during treatment with roflumilast (data not shown).

TABLE 3
Adverse events occurring in at least 2.5% of patients in one of the treatment groups

| | M2-124 | | | M2-125* | | |
|---|---|---|---|---|---|---|
| | Roflumilast | Placebo | Roflumilast vs placebo | Roflumilast | Placebo | Roflumilast vs placebo |
| | (n = 769)† | (n = 755)† | (difference, 95% CI) | (n = 778)‡ | (n = 790)‡ | (difference, 95% CI) |
| COPD | 70 (9%) | 82 (11%) | −1.76 (−4.90 to 1.38) | 87 (11%) | 122 (15%) | −4.26% (−7.74 to −0.78) |
| Diarrhoea | 63 (8%) | 26 (3%) | 4.75% (2.28 to 7.21) | 67 (9%) | 23 (3%) | 5.70% (3.28 to 8.12) |
| Weight loss | 92 (12%) | 24 (3%) | 8.78% (6.04 to 11.53) | 65 (8%) | 20 (3%) | 5.82% (3.46 to 8.18) |
| Nasopharyngitis | 57 (7%) | 50 (7%) | 0.79% (−1.91 to 3.49) | 35 (5%) | 47 (6%) | −1.45% (−3.78 to 0.88) |
| Upper respiratory | 16 (2%) | 21 (3%) | −0.70% (−2.38 to 0.98) | 33 (4%) | 38 (5%) | −0.57% (−2.75 to 1.62) |
| tract infection | | | | | | |
| Headache | 26 (3%) | 17 (2%) | 1.13% (−0.66 to 2.92) | 25 (3%) | 8 (1%) | 2.20% (0.65 to 3.75) |
| Pneumonia | 17 (2%) | 15 (2%) | 0.22% (−1.35 to 1.79) | 25 (3%) | 16 (2%) | 1.19% (−0.52 to 2.90) |
| Back pain | 27 (4%) | 22 (3%) | 0.60% (−1.30 to 2.50) | 23 (3%) | 13 (2%) | 1.31% (−0.30 to 2.92) |
| Acute Bronchitis | 35 (5%) | 40 (5%) | −0.75% (−3.05 to 1.56) | 21 (3%) | 24 (3%) | −0.34% (−2.12 to 1.44) |
| Nausea | 41 (5%) | 15 (2%) | 3.34% (1.34 to 5.35) | 21 (3%) | 15 (2%) | 0.80% (−0.81 to 2.41) |
| Hypertension | 20 (3%) | 28 (4%) | −1.11% (−2.99 to 0.78) | 18 (2%) | 20 (3%) | −0.22% (−1.87 to 1.43) |
| Insomnia | 19 (2%) | 8 (1%) | 1.41% (−0.04 to 2.86) | 18 (2%) | 12 (2%) | 0.79% (−069 to 2.28) |
| Decreased appetite | 21 (3%) | 2 (<1%) | 2.47% (1.13 to 3.81) | 15 (2%) | 5 (<1%) | 1.30% (0.05 to 2.54) |
| Influenza | 27 (4%) | 18 (2%) | 1.13% (−0.70 to 2.95) | 12 (2%) | 20 (3%) | −0.99% (−2.51 to 0.53) |

Data are number (%), unless otherwise indicated.
Adverse events were reported independently of the investigator causality assessments. Patients might have had more than on adverse event.
COPD = chronic obstructive pulmonary disease.
*Incidence of adverse events in roflumilast-treated patients in study M2-125 is in descending order.
†One patient was randomized twice, and included twice in the safety analysis but only once in the efficacy analysis; four patients assigned to placebo were given roflumilast instead and were included in the roflumilast group for the safety analysis; 765 patients in the roflumilast group and 758 in the placebo group were included in the efficacy analysis.
‡Six patients assigned to placebo were given roflumilast instead and were included in the roflumilast group for safety analysis;
772 patients in the roflumilast group and 796 in the placebo group were included in the efficacy analysis.

### Discussion

Roflumilast reduced exacerbation frequency and induced consistent and significant improvements in FEV₁, both before and after bronchodilator use. Similar changes occurred in FVC and mid-expiratory flow, suggesting a general improvement in operating lung volume. These changes were independent of the patient's smoking status or use of concomitant medication, such as inhaled long acting β2 agonists, and were similar to those reported in other patient populations with COPD. (References 14,19) PDE4 inhibition provides a novel approach to the treatment of patients with COPD. However, results from previous studies have shown inconsistent effects of PDE4 inhibitors on clinically relevant outcomes such as acute exacerbation frequency, although results from a post-hoc analysis suggested that roflumilast might be effective in selected patients with COPD (Reference 13). The results from the M2-124 and M2-125 studies show that carefully defined patient groups that are particularly at risk of exacerbations benefit from treatment with roflumilast. The effects of roflumilast in proposed subgroups, which should be easily identified clinically, were tested in these two adequately powered studies with an identical design, undertaken in two geographically different populations. Participants in both studies were preselected for specific characteristics identified from earlier trials (References 7, 19). They had substantial airflow limitation (stages III and IV according to the criteria of the Global initiative for chronic Obstructive Lung Disease), documented cough and sputum production as a marker for persistent airway inflammation, characterized as chronic bronchitis (Reference 20) and a history of exacerbations treated in the year before entry into the study.

As used herein, the term “chronic bronchitis” refers to chronic cough and sputum production. A clinical definition includes the presence of a chronic, productive cough for three months during each of two consecutive years (other causes of cough being excluded).

Many clinical trials identify patient subgroups that seem to respond to treatment in a secondary or post-hoc analysis, which is not confirmed in studies that are better powered. (Reference 21) In an earlier study, roflumilast did not reduce overall exacerbation rate but decreased the number of exacerbations requiring oral corticosteroids (Reference 14). Data from the present studies confirmed this finding. Treatment with inhaled corticosteroids has been shown to prevent exacerbations, including those that are subsequently managed with oral corticosteroids (References 7, 22). The same holds true for treatment with roflumilast. A direct comparison of the effect of inhaled steroids or roflumilast on reduction of exacerbations cannot be directly assessed with the present data, but is worth investigation in the future. The rate of exacerbations in our placebo-treated patients was higher than in previous studies, with few episodes being treated with antibiotics alone, possibly because of study design and patient recruitment.

As in other one year trials in patients with COPD, roflumilast did not have much effect on episodes requiring treatment in hospital, (References 23-25) which were infrequent. In the present studies, the number of patients needed to treat with roflumilast to prevent one exacerbation per year that was moderate or severe was 5.29 in the M2-124 study and 3.64 in the M2-125 study, irrespective of concurrent treatment with an inhaled long acting β2 agonist. Several secondary outcomes were assessed.

Mortality rate during treatment did not differ between treatments and was similar to other events during treatment in the first year of a large COPD survival trial. (Reference 7) The concentration of C-reactive protein was unaffected by treatment. However, the use of this marker in cardiorespiratory disease has been questioned. (Reference 26) Small but significant improvements in breathlessness assessed by the investigator-administered TDI occurred in both studies, but did not reach the agreed minimum clinically important difference. Whether this result indicates that the benefit of treatment with roflumilast is predominantly on prevention of exacerbations rather than improvement of exercise performance, or is a result of the selection criteria used will require further study.

Whether the effects of roflumilast are additive to long acting inhaled bronchodilators is discussed below (Reference 27). For practical reasons, the effect of roflumilast on breathlessness was tested rather than assessment of the global health status. In general, health status improves when the exacerbation rate falls by the magnitude seen here (References 28, 29), but confirmation of this association by means of a disease-specific instrument is needed for roflumilast. Changes in health status were not seen in the previous 1-year roflumilast study and the general health measure EQ-5D did not seem to identify differences in the data (Reference 14). The health-care utilization definition of exacerbations used in this study cannot precisely define the duration of events and might miss mild episodes (References 30-32). In other studies with daily diary cards, substantially more events have been identified than in our studies, including many events that were not treated with corticosteroids or antibiotics. The results of a previous study have suggested that mild events associated with increased symptoms and use of short acting β2 agonists could be prevented with roflumilast (Reference 19), the reduction in use of short acting β2 agonists that was noted in these studies supports this finding, Since roflumilast is an anti-inflammatory drug, the focus was on its ability to change corticosteroid-treated exacerbations. There were fewer antibiotic-treated episodes than expected, possibly indicating the way investigators interpreted the study protocol.

Interpretation of the data has been complicated by the pattern of patient withdrawal in these trials, which differed between treatment groups in the early and late phases. In general, this pattern would tend to result in a minimum biological effect of the active therapy by reducing the statistical power of the study comparisons. In accordance with good clinical trial practice, the focus was on recruiting patients likely to adhere to treatment and, thus, caution is needed when generalizing these findings to the general clinical population.

No significant neurological or cardiac toxicity was noted with roflumilast. A range of predicted adverse events occurred with roflumilast that were centrally mediated (insomnia, nausea, headache, but not vomiting) or gastrointestinal (predominantly diarrhea). These were most evident in the first 4-12 weeks of treatment when they contributed to the early difference in withdrawal in both studies. Thereafter, no difference was noted between treatment groups in the occurrence of these adverse events and the withdrawals associated with them. Patients reported weight loss more frequently in the roflumilast groups than in the placebo groups, a finding confirmed by objective measurements. The mean weight loss of 2.1 kg (SD 4.0) over the course of the study was greatest in the first 6 months of roflumilast treatment. Patients reporting gastrointestinal or neurological symptoms lost more weight, but weight loss was still seen in patients without these side-effects. The change in body weight was similar irrespective of initial BMI and might not be an unwelcome treatment effect in obese patients who showed the largest absolute weight loss.

The occurrence of pneumonias among patients in the roflumilast groups was not noted to be more than among those in the placebo groups, whereas pneumonia was reported more frequently with inhaled corticosteroids in studies with similar patient-years of treatment exposure to our studies (Reference 33). This increased frequency suggests that pneumonia might relate to local effects of inhaled corticosteroids rather than representing a general outcome of treatment with anti-inflammatory drugs in patients with COPD.

The present results from these clinical trials with identical design that were done in two different populations have shown that roflumilast, a PDE4 inhibitor, improves lung function and reduces the frequency of exacerbations in patients with bronchitic symptoms and severe airflow limitation. It should be noted that this treatment is not suitable for all patients because of the presence of class-related adverse effects that usually arise soon after initiation of treatment. Nonetheless, these results suggest that different subsets of patients exist within the broad range of COPD, and that specific therapies might improve disease management.

### Full Prescribing Information

In one embodiment of the present invention, the following full prescribing information would be provided to patients prescribed roflumilast for the maintenance treatment of chronic obstructive pulmonary disease associated with chronic bronchitis in patients at risk of exacerbations.

### Full Prescribing Information

### 1. Indications and Usage

Roflumilast (as used herein “roflumilast”, refers to the film coated tablet containing 500 micrograms of roflumilast, unless the context suggests otherwise) is indicated for the maintenance treatment of chronic obstructive pulmonary disease (COPD) associated with chronic bronchitis in patients at risk of exacerbations.

### 2. Dosage and Administration

The recommended dosage for patients with COPD is 1 tablet per day, with or without food.

### 3. Dosage Forms and Strengths

Yellow, D-shaped, film-coated tablet, embossed with “D” on one side, containing 500 micrograms (mcg) of roflumilast.

### 4. Contraindications

The use of roflumilast is contraindicated in patients with known hypersensitivity to any component of the formulation

### 5. Warnings and Precautions

### 5.1 Treatment of Acute COPD Symptoms

Roflumilast is an anti-inflammatory substance indicated for maintenance treatment of COPD. It is not indicated for the relief of acute bronchospasms.

### 5.2 Weight Decrease

In the event of an unexplained and pronounced weight decrease, patients should consult a healthcare professional. Further intake of roflumilast should be stopped, if deemed necessary. In 1-year studies (M2-124, M2-125), a decrease of body weight occurred in 62% of patients treated with roflumilast compared to 38% of placebo-treated patients. Weight decrease was irrespective of the BMI (body mass index). The mean absolute weight change over the 1-year period was −2.1 kg in roflumilast-treated patients. After discontinuation of roflumilast, the majority of patients had regained body weight after 3 months.

### 6. Adverse Reactions

The data described below reflect exposure to roflumilast in 6,563 COPD patients studied in placebo-controlled trials, including 4,138 exposed for up to 6 months, 1,193 exposed for up to one year, and 1,232 exposed for one year or longer. The population was 25 to 93 years old (median age 64), 72% were male, and 89% were white. Most patients received roflumilast doses of 500 mcg once daily.

Because clinical trials are conducted under widely varying conditions, adverse reaction rates observed in the clinical trials of a drug cannot be directly compared to rates in the clinical trials of another drug and may not reflect the rates observed in practice.

In general, roflumilast has been demonstrated to be well tolerated in short-term and long-term trials. The most common adverse reactions with incidence rates of 4% or more were: diarrhea, weight decrease, nausea and headache. These adverse reactions in patients on treatment with roflumilast in clinical trials mainly occurred within the first weeks of therapy and mostly resolved on continued treatment.

### 6.1 Adverse Reactions in Clinical Trials

The table below lists the adverse events reported in 1% or more of roflumilast-treated patients with COPD from 14 double-blind, placebo-controlled Phase II/III studies of up to 12 months that administered 250 mcg or 500 mcg roflumilast per day.

TABLE
Treatment-Emergent Adverse Events* Reported by ≧ 1% of Roflumilast-
Treated COPD Patients in 14 Phase II/III, Placebo-Controlled Studies

| % incidence | | |
|---|---|---|
| Roflumilast | | Placebo |
| Study event | (N = 6563) | (N = 5491) |
| Diarrhea | 10 | 3 |
| Weight decreased | 6 | 2 |
| Nausea | 5 | 1 |
| Headache | 4 | 2 |
| Back pain | 3 | 2 |
| Abdominal pain | 2 | 1 |
| Decreased appetite | 2 | 0 |
| Dizziness | 2 | 1 |
| Tremor | 2 | 0 |
| Insomnia | 2 | 1 |
| Gastritis | 1 | 0 |

*A treatment-emergent adverse event refers to any untoward medical event associated with the use of the drug in humans, whether or not considered drug-related, for which the incidence rate for roflumilast exceeds the rate for placebo.

Additional clinically significant treatment-emergent adverse reactions occurring in these clinical trials involving roflumilast with an incidence of less than 1% and at a greater incidence with roflumilast than with placebo include the following: abdominal discomfort, frequent bowel movements, asthenia, anorexia and pruritus.

### 7. Drug Interactions

A major step in roflumilast metabolism is the N-oxidation of roflumilast to roflumilast N-oxide by CYP 3A4 and CYP 1A2. Both roflumilast and roflumilast N-oxide have intrinsic phosphodiesterase 4 (PDE4) inhibitory activity. Therefore, following administration of roflumilast, the total PDE4 inhibition is considered to be the combined effect of both roflumilast and roflumilast N-oxide [See Clinical Pharmacology (12.3)]. No clinically relevant interactions with the following drugs were observed: inhaled salbutamol, formoterol, budesonide, oral theophylline, montelukast, digoxin, warfarin, sildenafil, midazolam and an oral contraceptive containing gestodene and ethinyl estradiol. Co-administration with an antacid did not alter the absorption or pharmacokinetics of roflumilast or its N-oxide.

### 7.1 Drugs That Inhibit Cytochrome P450 (CYP) Enzymes

Clinical drug-drug interaction studies with CYP 3A4 inhibitors (erythromycin and ketoconazole) did not result in increases of total PDE4 inhibitory activity (i.e. total exposure to roflumilast and roflumilast N-oxide). Interaction studies with CYP 1A2 inhibitor fluvoxamine, and dual CYP 3A4/1A2 inhibitors enoxacin and cimetidine resulted in increases in total PDE4 inhibitory activity. Therefore, an increase in the total PDE4 inhibition of 20% to 60% should be expected when roflumilast is concomitantly taken with strong CYP 1A2 inhibitors, such as fluvoxamine, while no alteration should be expected with strong CYP 3A4 inhibitors such as ketoconazole. No clinically relevant drug interactions are expected.

### 7.2 Drugs That Induce Cytochrome P450 (CYP) Enzymes

Administration of the cytochrome P450 enzyme inducer rifampicin resulted in a reduction in total PDE4 inhibitory activity by about 60% and use of strong cytochrome P450 inducers (e.g. phenobarbital, carbamazepine, phenyloin) may reduce the therapeutic effect of roflumilast.

### 8. Use in Specific Populations

8.1 Pregnancy

Pregnancy Category C: Roflumilast was not teratogenic in rats and rabbits following oral administration up to the highest doses of 1.8 mg/kg/day in rats and 0.8 mg/kg/day in rabbits. Administered at the same doses, roflumilast has been shown to induce mild retardation of embryo-fetal development (incomplete ossification) in the rat, but not in the rabbit. Exposure of pregnant rats to unbound roflumilast and roflumilast N-oxide was 1.7 and 10.8 times higher, respectively, than exposure of women at the 500 mcg roflumilast dose. In one of three rat studies on fertility and embryo-fetal-development, post-implantive losses were observed at oral doses of 0.6 mg/kg/day and 1.8 mg/kg/day. Post-implantive losses were not seen in rabbits up to doses of 0.8 mg/kg/day. Rat and rabbit fetuses were exposed to roflumilast and the permeability of the placental barrier for drug-related material increased with the progression of pregnancy. There are no adequate and well-controlled studies of roflumilast in pregnant women. Data on a limited number (20) of exposed pregnancies indicate no adverse effects of roflumilast on pregnancy or on the health of the fetus or new-born child. Nonetheless, the safe use during pregnancy is not established and roflumilast should not be used during pregnancy.

### 8.2 Labor and Delivery

Signs of tocolytic activity resulting in delivery retardation and decreased postnatal survival were observed in the mouse at oral doses of 2 mg/kg/day and above. There are no human studies that have investigated effects of roflumilast on preterm labor or labor at term. Roflumilast should not be used during labor and delivery.

### 8.3 Nursing Mother

Roflumilast and/or its metabolites are excreted into the milk of lactating rats. Excretion of roflumilast and/or metabolites into human milk is probable. Roflumilast should not be used during breast-feeding.

### 8.4 Pediatric Use

Safety and effectiveness of roflumilast in children and adolescents below 18 years of age have not been established. Roflumilast is not recommended in this population.

### 8.5 Geriatric Use

In clinical studies with roflumilast, there were no overall differences in safety and effectiveness of roflumilast in the elderly compared to younger patients with COPD. Therefore, no dose adjustment is necessary [See Clinical Pharmacology (12.3)].

### 8.6 Hepatic Impairment

The pharmacokinetics of roflumilast 250 mcg once-daily was tested in patients with mild-to-moderate hepatic impairment classified as Child-Pugh A and B. In these patients, the total PDE4 inhibitory activity was increased by about 30% in patients with Child-Pugh A and about 50% in patients with Child-Pugh B. Simulations suggest dose proportionality between roflumilast 250 and 500 mcg in patients with mild-to-moderate hepatic impairment. Therefore, no dose adjustment is necessary in these patients. The pharmacokinetics of roflumilast in patients with severe hepatic impairment (Child-Pugh Class C) was not tested, and therefore the use of roflumilast is not recommended in these patients [See Clinical Pharmacology (12.3)].

### 8.7 Renal Impairment

Total PDE4 inhibitory activity was decreased by 9% in patients with severe renal impairment (creatinine clearance 10-30 mL/min). No dose adjustment is necessary [See Clinical Pharmacology (12.3)].

### 10. Overdosage

### 10.1 Human Experience

No case of overdose has been reported in clinical studies with roflumilast. During the Phase I studies of roflumilast the following symptoms were observed at an increased rate after single oral doses of 2,500 mcg and one single dose of 5,000 mcg (ten times the recommended dose): headache, gastrointestinal disorders, dizziness, palpitations, lightheadedness, clamminess and arterial hypotension.

### 10.2 Management of Overdose

In case of overdose, patients should seek immediate medical help. Appropriate supportive medical care should be provided. Since roflumilast is highly protein bound, haemodialysis is not likely to be an efficient method of drug removal. It is not known whether roflumilast is dialyzable by peritoneal dialysis.

### 11. Description

The active ingredient in roflumilast film-coated tablets is roflumilast. The chemical name of roflumilast is N-(3,5-dichloropyridin-4-yl)-3-cyclopropylmethoxy-4-difluoromethoxy-benzamide. Its empirical formula is C₁₇H₁₄C₁₂F₂N₂O₃ and the molecular weight is 403.22.

The chemical structure is:

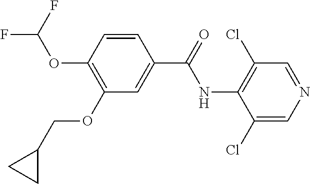

The drug substance is poorly soluble in water.

Roflumilast is supplied as a yellow, D-shaped film-coated tablet, embossed with “D” on one side that contains 500 mcg of roflumilast. Each film-coated tablet of roflumilast for oral administration contains the following inactive ingredients: lactose monohydrate, maize starch, povidone and magnesium stearate. In addition, the film-coat contains: hypromellose, Macrogol 4000, titanium dioxide and yellow iron oxide.

### 12. Clinical Pharmacology

### 12.1 Mechanism of Action

Roflumilast is a phosphodieserase 4 (PDE4) inhibitor. It is a non-steroid, anti-inflammatory agent designed to target both the systemic and pulmonary inflammation associated with chronic obstructive pulmonary disease. The mechanism of anti-inflammatory action of roflumilast is the inhibition of PDE4, a major cAMP-metabolizing enzyme found in structural and inflammatory cells important to the pathogenesis of COPD. Roflumilast targets the PDE4A, 4B and 4D splicing variants with similar potency in the nanomolar range. The affinity to the PDE4C splicing variants is 5 to 10-fold lower. This mechanism of action and the selectivity also apply to roflumilast N-oxide, which is the major active metabolite of roflumilast.

### 12.2 Pharmacodynamics

Inhibition of PDE4 leads to elevated intracellular cAMP levels and mitigates COPD-related malfunctions of leukocytes, airway and pulmonary vascular smooth muscle cells, endothelial and airway epithelial cells and fibroblasts. Based on this mechanism, roflumilast in experimental animals suppressed the release of inflammatory mediators, i.e. cytokines and reactive oxygen species from cells and lung tissue in vitro and in vivo. In addition, roflumilast inhibited the infiltration of leukocytes, in particular neutrophils, into the lungs of experimental animals. Roflumilast also reduced the smoke-induced destruction of lung parenchyma and prevented lung fibrotic and vascular remodeling in animal models in vivo. It stimulated bronchial ciliary activity in vitro and inhibited the formation of MUC5AC, a goblet cell-derived gel-forming mucin, in human airway epithelial cells and in animal experiments. These effects also apply to roflumilast N-oxide and respective in vitro and in vivo data concur with its PDE4 inhibitory potency. In patients with COPD, roflumilast reduced sputum neutrophils. Furthermore, roflumilast attenuated influx of neutrophils and eosinophils into the airways of endotoxin challenged healthy volunteers.

### 12.3 Pharmacokinetics

Roflumilast is extensively metabolized in humans, with the formation of a major pharmacodynamically active metabolite, roflumilast N-oxide. Since both roflumilast and roflumilast N-oxide contribute to PDE4 inhibitory activity in vivo (see Biotransformation below), pharmacokinetic considerations are based on total PDE4 inhibitory activity (i.e. total exposure to roflumilast and roflumilast N-oxide).

### Absorption

The absolute bioavailability of roflumilast following a 500 mcg oral dose is approximately 80%. Maximum plasma concentrations of roflumilast typically occur approximately one hour after dosing (ranging from 0.5 to 2 hours) in the fasted state while plateau-like maximum concentrations of the N-oxide metabolite are reached after about eight hours (ranging from 4 to 13 hours). Cₘₐₓ for roflumilast is 8.8 ug/L and 8.5 ug/L for the N-oxide metabolite. Food intake does not affect the total PDE4 inhibitory activity, but delays time to maximum concentration (tₘₐₓ) of roflumilast by one hour and reduces Cₘₐₓ by approximately 40%. However, C. and tₘₐₓ of roflumilast N-oxide are unaffected.

### Distribution

Plasma protein binding of roflumilast and its N-oxide metabolite is approximately 99% and 97%, respectively. Volume of distribution for single dose 500 mcg roflumilast is about 2.9 L/kg. Studies in rats with radiolabeled roflumilast indicate low penetration across the blood-brain barrier.

### Biotransformation

Roflumilast is extensively metabolized via Phase I (cytochrome P450) and Phase II (conjugation) reactions. The N-oxide metabolite is the major metabolite observed in the plasma of humans. The plasma AUC of the N-oxide metabolite on average is about 10-fold greater than the plasma AUC of roflumilast (315 ugxh/L for the N-oxide metabolite and 28 ugxh/L for roflumilast). Thus, the N-oxide metabolite is considered to be the main contributor to the total PDE4 inhibitory activity in vivo. As used herein, a roflumilast formulation having a rate and extent of absorption of falling within a range of −20%/+25% (and within a 90% confidence interval) of the AUC and Cₘₐₓ values disclosed above, would be considered bioequivalent to the formulation described herein. In vitro studies and clinical drug-drug interaction studies suggest that the metabolism of roflumilast to its N-oxide metabolite is mediated by CYP 1A2 and 3A4. Based on further in vitro results in human liver microsomes, therapeutic plasma concentrations of roflumilast and roflumilast N-oxide do not inhibit CYP 1A2, 2A6, 2B6, 2C8, 2C9, 2C19, 2D6, 2E1, 3A4/5, or 4A9/11. Therefore, there is a low probability of relevant interactions with substances metabolized by these P450 enzymes. In addition, in vitro studies demonstrated no induction of the CYP 1A2, 2A6, 2C9, 2C19, or 3A4/5 and only a weak induction of CYP 2B6 by roflumilast.

### Elimination

The plasma clearance after short-term intravenous infusion of roflumilast is on average about 9.6 L/h. Following an oral dose, the median plasma effective half-life of roflumilast and its N-oxide metabolite are approximately 17 and 30 hours, respectively. Steady state plasma concentrations of roflumilast and its N-oxide metabolite are reached after approximately 4 days for roflumilast and 6 days for roflumilast N-oxide following once daily dosing. Following intravenous or oral administration of radiolabeled roflumilast, about 70% of the radioactivity was recovered in the urine.

### Linearity/Non-Linearity

The pharmacokinetics of roflumilast and its N-oxide metabolite are dose-proportional over a range of doses from 250 mcg to 1,000 mcg.

### Special Populations—

### Renal Impairment

In patients with severe renal impairment, total PDE4 inhibitory activity was slightly decreased. These differences are not considered to be clinically relevant. Dosage modifications are not required.

### Hepatic Impairment

In patients with mild-to-moderate hepatic impairment classified as Child-Pugh A or B total PDE4 inhibitory activity was increased. These differences are not considered to be clinically relevant. Dosage modifications are not required in patients with mild-to-moderate hepatic impairment. There are no data on the pharmacokinetics of roflumilast in patients with severe hepatic impairment (Child-Pugh C). Therefore, roflumilast is not recommended in patients with severe hepatic impairment.

### Age

In the elderly, total PDE4 inhibitory activity was increased. These differences are not considered to be clinically relevant. Dosage modifications are not required.

### Gender

In women, total PDE4 inhibitory activity was increased when compared with men. These differences are not considered to be clinically relevant. Dosage modifications are not required.

### Smoking

In smokers, total PDE4 inhibitory activity was slightly decreased. However, the effectiveness was comparable irrespective of the current smoking status.

### Race

In African-Americans and Hispanics, simulations suggest that total PDE4 inhibitory activity is higher than in Caucasians. These differences are not considered to be clinically relevant. Dosage modifications are not required.

### 13. Nonclinical Toxicology

### 13.1 Carcinogenesis, Mutagenesis, Impairment of Fertility

### Carcinogenesis

Roflumilast was administered by gavage to male and female B6C3F1 mice at doses up to 12 mg/kg/day (males), and 18 mg/kg/day (females) over two years. No compound-related tumors occurred. In the two year hamster carcinogenicity studies at doses up to 16 mg/kg/day, nasal neoplasms were caused by a drug metabolite, which is absent in humans. No other treatment-related neoplastic findings were observed. Overall, the tumor-free level in the animals was 4 mg/kg/day.

### Mutagenesis

Roflumilast did not reveal a genotoxic potential in a standard battery of genotoxicity assays in vitro and in vivo assessing different genetic endpoints.

### Impairment of Fertility

There was no effect on female fertility up to the highest roflumilast dose of 1.5 mg/kg/day in rats. Slight reduction in male fertility was seen in conjunction with epididymal toxicity in rats dosed with 1.8 mg/kg/day (about 2.2 and 8.8 times human exposure to unbound roflumilast and roflumilast N-oxide, respectively). No epididymal toxicity or changes in semen parameters or fertility was present in any other rodent or non-rodent species including monkeys in spite of higher drug exposure. In a human spermatogenesis study, roflumilast 500 mcg had no effects on semen parameters or reproductive hormones during the 3-month treatment period and the following 3-month off-treatment period.

13.2 Animal Toxicology and/or Pharmacology

Non-clinical data reveal no special hazard for humans based on conventional studies of safety pharmacology and repeated dose toxicity. Furthermore, there is no evidence of immunotoxic, skin sensitizing or phototoxic potential.

### 14. Clinical Studies

### 14.1 Chronic Obstructive Pulmonary Disease (COPD)

The main clinical registration program consisted of two replicate one-year trials (M2-124 and M2-125) and two supplementary six-month trials (M2-127 and M2-128), all randomized, parallel-design, double-blind and placebo-controlled, with a total number of 4,768 randomized and treated patients of whom 2,374 were treated with roflumilast. Studies M2-124 and M2-125 included patients with a history of COPD associated with chronic bronchitis for at least 12 months prior to baseline, with symptoms at baseline as determined by cough and sputum score, non-reversible airway obstruction (FEV₁/FVC ratio of ≦70%), an FEV₁≦50% of predicted and at least one documented COPD exacerbation in the previous year.

In the one-year trials, long-acting beta-2 agonists (LABA) were allowed and used in approximately 50% of the study population. The use of inhaled corticosteroids was terminated at randomization. Lung function (pre-bronchodilator forced expiratory volume in one second, FEV₁) and the rate of moderate exacerbations (requiring intervention with systemic glucocorticosteroids) or severe exacerbations (resulting in hospitalization and/or leading to death) were primary endpoints. Secondary endpoints in both studies included further evaluation of exacerbations and lung function parameters, dyspnea, and use of reliever medication.

In a pooled analysis of the replicate one-year studies M2-124 and M2-125, roflumilast 500 mcg once daily significantly improved lung function compared to placebo, on average by 48 mL (pre-bronchodilator FEV₁, primary endpoint, p<0.0001), and by 55 mL (post-bronchodilator FEV₁, p<0.0001). Pre-bronchodilator forced vital capacity (FVC) was significantly greater with roflumilast than placebo in both studies by 89 mL (p<0.0001) in M2-124 and 108 mL (p<0.0001) in M2-125. Similar significant improvements were seen in post-bronchodilator FVC and pre-bronchodilator mid-expiratory flow. These changes in lung function were similar irrespective of concomitant treatment with or without LABA. In the pooled analysis, roflumilast 500 mcg increased mean pre-bronchodilator FEV₁ by 46 mL (p<0.0001), as compared to placebo with concomitant LABA treatment, and by 50 mL (p<0.0001) without concomitant LABA treatment.

In a pooled analysis, the endpoint of moderate or severe exacerbations was reduced by 17% (primary endpoint; p=0.0003). The numbers of patients needed to treat (NNT) to avoid one moderate or severe exacerbation per patient per year were 5.3 (M2-124) and 3.6 (M2-125). The number of patients experiencing a moderate exacerbation in the roflumilast group was 624 vs. 723 in the placebo group (Risk Ratio: 0.88; p=0.0011). The number of patients experiencing a severe exacerbation in the roflumilast group was 157 vs. 198 in the placebo group (Risk Ratio: 0.84; p=0.0715). Furthermore, the transitional dyspnea index (TDI) improved with roflumilast 500 mcg by on average 0.25 (p<0.0009) as compared to placebo.

The six-month studies M2-127 and M2-128 included patients with a history of COPD for at least 12 months prior to baseline. In study M2-128 in addition, documentation of chronic bronchitis and high reliever medication use was required. Both studies included patients with a non-reversible airway obstruction (FEV₁/FVC<70%) and a FEV₁ of 40% to 70% of predicted. Roflumilast or placebo treatment was added to continuous treatment with a long-acting bronchodilator, in particular salmeterol in study M2-127 or tiotropium in study M2-128.

In these two studies, pre-bronchodilator FEV₁ was significantly improved by 49 mL (primary endpoint, p<0.0001) beyond the bronchodilator effect of concomitant treatment with salmeterol in study M2-127 and by 80 mL (primary endpoint, p<0.0001) incremental to concomitant treatment with tiotropium in study M2-128. The corresponding post-bronchodilator values were 60 mL (p<0.0001) and 81 mL (p<0.0001) in studies M2-127 and M2-128, respectively.

These six-month studies were neither designed nor powered to show a statistically significant effect on exacerbations. However, analysis of data indicated that the reduction in the rate of moderate or severe exacerbations with roflumilast reached the level of statistical significance in study M2-127 (reduction by 37%; p=0.0315, post-hoc analysis), but not in study M2-128 (reduction by 23%; p=0.1957). In study M2-128 the TDI focal score improved in roflumilast treated patients by 0.4 (p=0.0032) beyond the bronchodilator effect of tiotropium.

In both the one-year and six-month studies, the improvement in lung function was sustained over the treatment period. Improvements in lung function and reduction of exacerbations was independent of underlying treatment with long-acting bronchodilators. Smoking status did not influence the improvement in lung function or reduction in exacerbations. Effects were similar, independent of previous treatment with inhaled corticosteroids.

In a pooled post-hoc analysis of two previous one-year studies (M2-111 and M2-112) including patients with a history of COPD associated with chronic bronchitis and emphysema, improvements in lung function was also shown to be independent of concomitant treatment with inhaled corticosteroids.

### 16. How Supplied/Storage and Handling

### 16.1 How Supplied

Roflumilast is supplied as 500 mcg yellow, D-shaped film-coated tablets, embossed with “D” on one side. Roflumilast tablets are available in polyethylene (PE) bottles with a polypropylene (PP) screw cap containing 30 tablets or 90 tablets.

### 16.2 Storage and Handling

Store roflumilast 500 mcg film-coated tablets at 20°-25° C. (68°-77° F.); excursions permitted to 15°-30° C. (59°-86° F.). [See USP Controlled Room Temperature]. The drug product shelf life is 24 months. Over a 24 month shelf life, the roflumilast tablets produce by degradation, N-(3,5-dichloro-pyridin-4-yl)-4-difluoromethoxy-3-hydroxybenzamide and N-(3,5-dichloropyridin-4-yl)-3cyclopropyl-methoxy-4-hydroxybenzamide, in amounts of less than 1.0% by weight, and preferably less than 0.15%, each, and most preferably, less than 0.15% cumulatively by weight, of the total amount of roflumilast present in the tablets.

### 17. Patient Counseling Information

### 17.1 Not for Acute Bronchospasms

Patients should be informed that roflumilast is indicated for maintenance treatment of COPD. It is not indicated for the relief of acute bronchospasms.

### 17.2 Weight Decrease

Patients should be informed that in the event of an unexplained and pronounced weight decrease, they should consult a healthcare professional.

### REFERENCES

1 Kessler R, Stahl E, Vogelmeier C, et al. Patient understanding, detection, and experience of COPD exacerbations: an observational, interview-based study. Chest 2006; 130: 133-42.
2 Donaldson G C, Seemungal T A, Bhowmik A, Wedzicha J A. Relationship between exacerbation frequency and lung function decline in chronic obstructive pulmonary disease. Thorax 2002; 57: 847-52.
3 Soler-Cataluna J J, Martinez-Garcia M A, Roman S P, Salcedo E, Navarro M, Ochando R. Severe acute exacerbations and mortality in patients with chronic obstructive pulmonary disease. Thorax 2005; 60:925-31.
4 Spencer S, Calverley P M, Sherwood B P, Jones P W. Health status deterioration in patients with chronic obstructive pulmonary disease. Am J Respir Crit Care Med 2001; 163: 122-28.
5 Rabe K F, Hurd S, Anzueto A, et al. Global strategy for the diagnosis, management, and prevention of chronic obstructive pulmonary disease: GOLD executive summary. Am J Respir Crit. Care Med 2007; 176: 532-55.
6 Brusasco V, Hodder R, Miravitlles M, Korducki L, Towse L, Kesten S. Health outcomes following treatment for six months with once daily tiotropium compared with twice daily salmeterol in patients with COPD. Thorax 2003; 58: 399-404.
7 Calverley P M, Anderson J A, Celli B, et al. Salmeterol and fluticasone propionate and survival in chronic obstructive pulmonary disease. N Engl J Med 2007; 356: 775-89.
8 Stockley R A, Chopra N, Rice L. Addition of salmeterol to existing treatment in patients with COPD: a 12 month study. Thorax 2006; 61: 122-28.
9 Tashkin D P, Celli B, Senn S, et al. A 4-year trial of tiotropium in chronic obstructive pulmonary disease. N Engl J Med 2008; 359: 1543-54.
10 Bundschuh D S, Eltze M, Barsig J, Wollin L, Hatzelmann A, Beume R. In vivo efficacy in airway disease models of roflumilast, a novel orally active PDE4 inhibitor. J Pharmacol Exp Ther 2001; 297: 280-90.
11 Hatzelmann A, Schudt C. Anti-inflammatory and immunomodulatory potential of the novel PDE4 inhibitor roflumilast in vitro. J Pharmacol Exp Ther 2001; 297: 267-79.
12 Spina D. PDE4 inhibitors: current status. Br J Pharmacol 2008; 155: 308-15.
13 Grootendorst D C, Gauw S A, Verhoosel R M, et al. Reduction in sputum neutrophil and eosinophil numbers by the PDE4 inhibitor roflumilast in patients with COPD. Thorax 2007; 62: 1081-87.
14 Calverley P M, Sanchez-Toril F, Mclvor A, Teichmann P, Bredenbroeker D, Fabbri L M. Effect of 1-year treatment with roflumilast in severe chronic obstructive pulmonary disease. Am J Respir Crit. Care Med 2007; 176: 154-61.
15 Mahler D A, Weinberg D H, Wells C K, Feinstein A R. The measurement of dyspnea. Contents, interobserver agreement, and physiologic correlates of two new clinical indexes. Chest 1984; 85: 751-58.
16 Sin D D, Man S F. Skeletal muscle weakness, reduced exercise tolerance, and COPD: is systemic inflammation the missing link? Thorax 2006; 61: 1-3.
17 Rabin R, de CF. EQ-5D: a measure of health status from the EuroQol Group. Ann Med 2001; 33: 337-43.
18 Verbeke G, Molenberghs G. Linear mixed models for longitudinal data. Series in Statistics. New York: Springer, 2000.
19 Rabe K F, Bateman E D, O'Donnell D, Witte S, Bredenbröker D, Bethke T D. Roflumilast—an oral anti-inflammatory treatment for chronic obstructive pulmonary disease: a randomised controlled trial. Lancet 2005; 366: 563-71.
20 Snoeck-Stroband J B, Lapperre T S, Gosman M M, et al. Chronic bronchitis sub-phenotype within COPD: inflammation in sputum and biopsies. Eur Respir J 2008; 31: 70-77.
21 Assmann S F, Pocock S J, Enos L E, Kasten L E. Subgroup analysis and other (mis)uses of baseline data in clinical trials. Lancet 2000; 355: 1064-69.
22 Lanes S F, Jara M. The INSPIRE study: influence of prior use and discontinuation of inhaled corticosteroids. Am J Respir Crit. Care Med 2008; 178: 543-44.
23 Calverley P, Pauwels R, Vestbo J, et al. Combined salmeterol and fluticasone in the treatment of chronic obstructive pulmonary disease: a randomised controlled trial. Lancet 2003; 361: 449-56.
24 Calverley P M, Boonsawat W, Cseke Z, Zhong N, Peterson S, Olsson H. Maintenance therapy with budesonide and formoterol in chronic obstructive pulmonary disease. Eur Respir J 2003; 22: 912-19.
25 Calverley P M, Rennard S, Nelson H S, et al. One-year treatment with mometasone furoate in chronic obstructive pulmonary disease. Respir Res 2008; 9: 73.
26 Casas J P, Shah T, Hingorani A D, Danesh J, Pepys M B. C-reactive protein and coronary heart disease: a critical review. J Intern Med 2008; 264: 295-314.
27 Fabbri L M, Calverley P M, Izquierdo-Alonso J L, et al, for the M2-127 and M2-128 study groups. Roflumilast in moderate-to-severe chronic obstructive pulmonary disease treated with long-acting bronchodilators: two randomised clinical trials. Lancet 2009; 374: 695-703.
28 Burge P S, Calverley P M A, Jones P W, Spencer S, Anderson J A, Maslen T K. Randomised, double blind, placebo controlled study of fluticasone propionate in patients with moderate to severe chronic obstructive pulmonary disease: the ISOLDE trial. BMJ 2000; 320: 1297-303.
29 Calverley P M, Walker P. Chronic obstructive pulmonary disease. Lancet 2003; 362: 1053-61.
30 Keene O N, Calverley P M, Jones P W, Vestbo J, Anderson J A. Statistical analysis of exacerbation rates in COPD: TRISTAN and ISOLDE revisited. Eur Respir J 2008; 32: 17-24.
31 Keene O N, Calverley P M, Jones P W, Vestbo J, Anderson J A. Statistical analysis of COPD exacerbations. Eur Respir J 2008; 32: 1421-22.
32 Suissa S. Exacerbations and intent-to-treat analyses in randomised trials. Eur Respir J 2008; 32: 1117-18.
33 Wedzicha J A, Calverley P M, Seemungal T A, Hagan G, Ansari Z, Stockley R A. The prevention of chronic obstructive pulmonary disease exacerbations by salmeterol/fluticasone propionate or tiotropium bromide. Am J Respir Crit. Care Med 2008; 177: 19-26.

### Salmeterol+Roflumilast and Tiotropium+Roflumilast Studies

To determine whether roflumilast provides benefit to patients who are regularly treated with longacting inhaled bronchodilators, its effects in patients with COPD who were regularly treated with salmeterol or tiotropium was investigated.

The salmeterol plus roflumilast (M2-427) trial was done in 135 centers in ten countries, whereas the tiotropium plus roflumilast (M2-128) trial was done in 85 centers in seven countries.

Patients with moderate-to-severe COPD, which was defined spirometrically, were recruited from an outpatient setting to investigate the effect of roflumilast concomitantly with salmeterol or tiotropium. The main inclusion criteria were age older than 40 years, current or former smokers (≧1 year of smoking cessation) with a smoking history of at least ten pack-years, postbronchodilator forced expiratory volume in 1 s (FEV₁) of 40-70% of predicted value, a postbronchodilator FEV₁ to forced vital capacity (FVC) ratio of less than or equal to 0.70, partial reversibility to albuterol (400 μg; increase in baseline FEV₁ of ≦12% or 200 mL), and stable disease.

Predicted values for FEV₁, FVC and FEV₁/FVC, as used in the M2-127 and M2-128 studies, were calculated according to the formula of the European Community for Coal and Steel (ECCS). Quanjer P. H., et al., Lung Volumes and Forced Ventilatory Flows. Report Working Party Standardization of Lung Function Tests, Eur Respir J 1993; 16 (suppl): 5-40.

| | | |
|---|---|---|
| FEV₁ [L] | male | (4.30 × height [m]) − (0.029 × age [y]) − 2.49 |
| | female | (3.95 × height [m]) − (0.025 × age [y]) − 2.60 |
| FVC [L] | male | (5.76 × height [m]) − (0.026 × age [y]) − 4.34 |
| | female | (4.43 × height [m]) − (0.026 × age [y]) − 2.89 |
| FEV₁/FVC | male | (−0.18 × age [y]) + 87.21 |
| | female | (−0.19 × age [y]) + 89.10 |

For non-Caucasians (Black) predicted values for FEV₁ and FVC are obtained by multiplying values by a factor of 0.9).

By contrast with the salmeterol plus roflumilast trial, patients recruited to the tiotropium plus roflumilast trial were more symptomatic because they had to have chronic cough and sputum production, and frequent use of as-needed shortacting β2 agonists (at least 28 puffs per week) during the run-in period while they were being treated with tiotropium for at least 3 months before enrolment.

In an initial, 4-week run-in, patients in both studies were given placebo tablets once a day in the morning. They recorded their use of shortacting bronchodilators, and cough and sputum production on daily diary cards, In this initial study phase, patients, but not investigators, were unaware of the treatment they were assigned to. Patients who took at least 80% of prescribed placebo tablets without evidence of a moderate or severe exacerbation of COPD during the run-in period were randomly assigned to roflumilast 500 μg once a day in the morning or placebo for the subsequent 24 weeks.

In the double-blind treatment phase, all individuals involved in the studies were unaware of treatment assignment. The investigator or anyone at the study site was prevented from knowing the allocation sequence with code labelling—tablets were identical in appearance. The sponsor and clinical research associate were notified if there was a clinical reason for an individual's treatment to be unmasked by the investigator with the interactive voice recognition system.

Besides salmeterol or tiotropium, no inhaled corticosteroids, shortacting anticholinergic drugs, other longacting bronchodilator drugs, theophylline, or other respiratory drugs were allowed after study enrolment,

After randomization, patients were assessed every 1 weeks up to week 12, and every 6 weeks thereafter until week 24. At each visit, spirometric measurements were recorded before and 30 min after administration of bronchodilator (inhaled albuterol 400 μg). Additionally, any new exacerbations or adverse events were recorded, the patient's bodyweight, adherence to taking tablets, completeness of the daily diary records, use of shortacting β2 agonists, and investigator-administered transition dyspnoea index (TDI) and Shortness of Breath Questionnaire (SOBQ), and dispensed study medication. Exacerbations were defined as mild if the patient needed an increase in rescue medication of at least three puffs per day on at least 2 consecutive days during the double-blind treatment period; moderate if the patient needed oral corticosteroids (not antibiotics); and severe if the patient needed treatment in hospital or died.

The primary endpoint in both studies was change in mean prebronchodilator FEV₁ from baseline to each postrandomisation visit. Secondary endpoints in both trials included postbronchodilator FEV₁ and FVC, TDI score, SOBQ, rate of COPD exacerbations, and use of rescue medications.

At each visit, safety assessments included inquiries about the occurrence of adverse events. Bodyweight was measured with the same scales at each visit, height was measured with a stadiometer, and body-mass index (BMI) was calculated. At baseline and 24 weeks after randomization, blood samples were taken for routine hematology and biochemistry tests and measurements of C-reactive protein (a possible marker of systemic inflammation in COPD), and an electrocardiogram (ECG) was done.

All reported data analyses were pre-specified, and data are presented as mean and SD, unless otherwise indicated. Data for efficacy were evaluated with an intention-to-treat analysis in patients given at least one dose of study medication. Both studies were powered for the primary endpoint—i.e., change in prebronchodilator FEV₁ from baseline, which was analyzed by repeated-measures analysis of covariance.

The assumptions made for the primary endpoint on the basis of data gathered in a previous study were compound symmetry structure with equal variance (common SD of 240 mL) for all five time points and both treatments, equal correlation of 0.6 between all pairs of time points for each patient, and normally distributed changes from baseline. The estimate of the treatment effect (50 mL) was based on clinical considerations and was in agreement with previous studies of inhaled glucocorticosteroids added to longacting β2 agonists. The sample size was calculated for the repeated-measures analysis of covariance model according to Chow and colleagues. On the basis of assumptions outlined above and the use of a one-sided significance level of 2.5%, the power was 97% with a sample size of 469 patients per treatment group in the salmeterol plus roflumilast trial, and the power was 91% with a sample size of 350 patients per treatment group in the tiotropium plus roflumilast trial. The salmeterol plus roflumilast trial was originally powered for a traditional analysis of covariance model and not for a repeated-measures analysis of covariance model. After completion of recruitment, but before unmasking the studies, the statistical analysis model was changed to the more powerful repeated-measures analysis of covariance model, accounting for the larger number of patients recruited and the higher statistical power in the salmeterol plus roflumilast trial than in the tiotropium plus roflumilast trial. A conservative approach was taken for the main analysis of the repeated measurements of expiratory lung function variables, patient diary variables, SOBQ, and TDI scores, and no missing values were replaced in these two trials.

In both studies, the repeated-measures analysis of covariance model included the factors and covariates of treatment, value at baseline, age, sex, smoking status, country, time, and treatment-by-time interaction. Several statistical analyses were preplanned and done with the intent to assess the robustness of results with respect to the effect of differential dropouts and missing data. Adverse events were analyzed with descriptive statistics and 95% CIs for the differences between treatment groups. The natural log-transformed C-reactive protein concentration (mean change from baseline to study end) was used for statistical analysis.

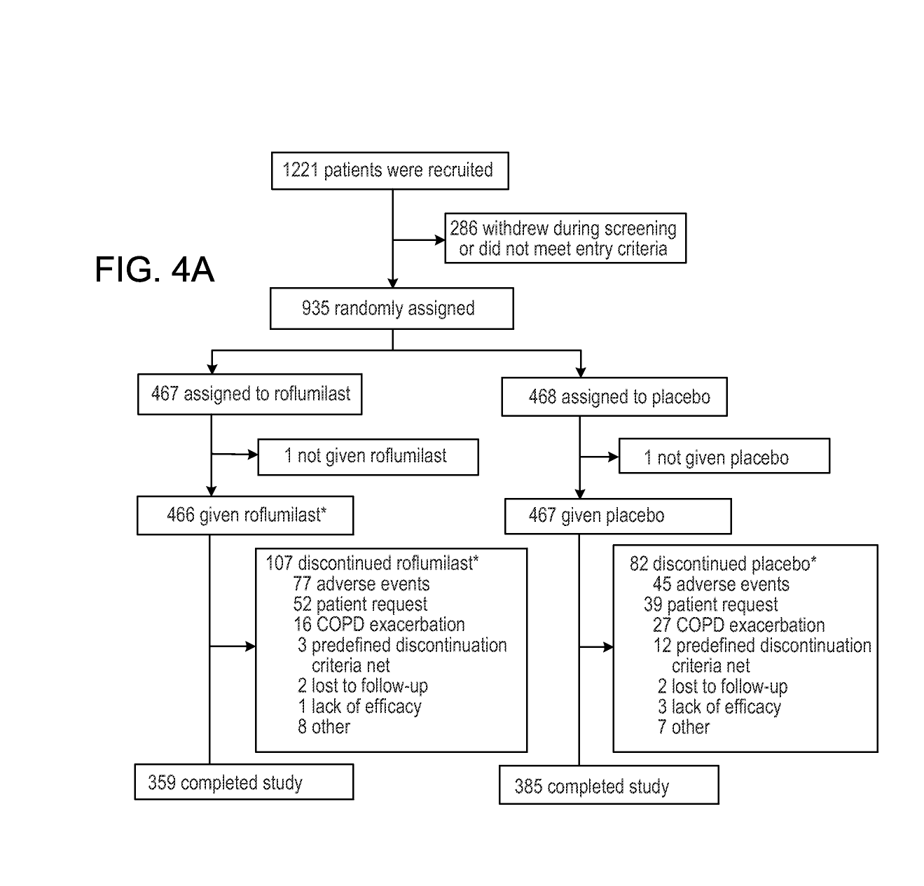

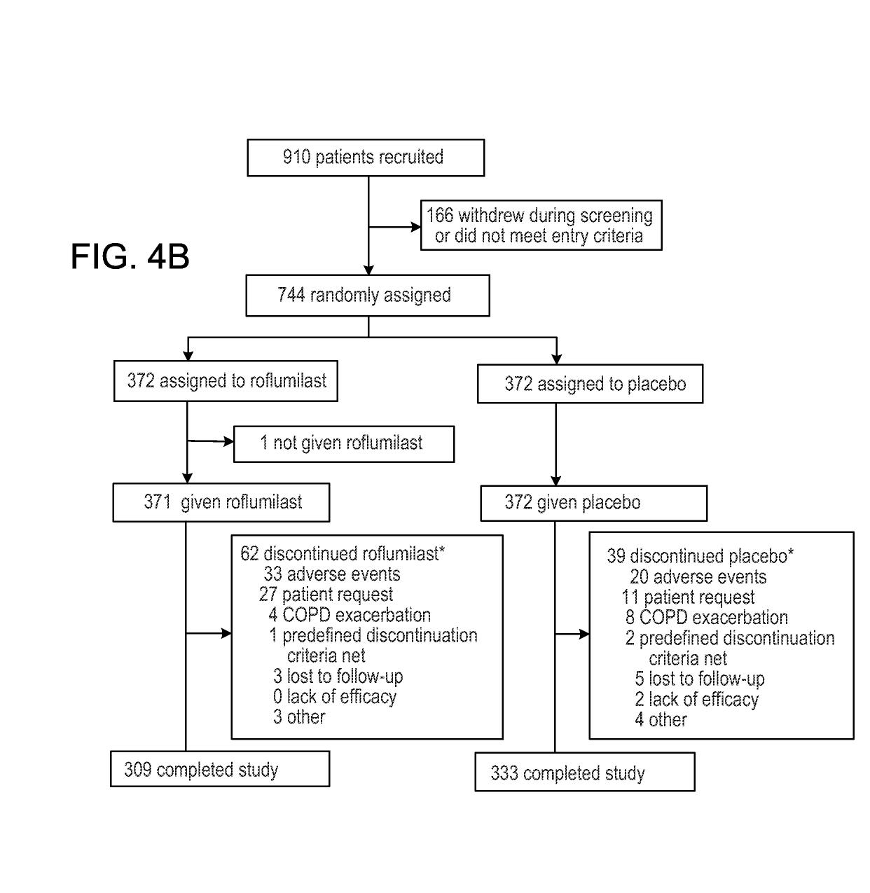

In the salmeterol plus roflumilast trial, 933 patients were randomly assigned and treated; 744 patients completed the study (FIG. 4A). In the tiotropium plus roflumilast trial, 743 patients were randomly assigned and treated, and 642 completed the study (FIG. 4B). Table 4 shows the demographic and baseline characteristics of the two intention-to-treat study populations.

TABLE 4
Baseline characteristics of the intention to treat populations assessed in the salmeterol plus roflumilast
(M2-127) and tiotropium plus roflumilast (M2-128) trials

| | M2-127 | | M2-128 | |
|---|---|---|---|---|
| | Salmeterol + roflumilast | Salmeterol + placebo | Tiotropium + roflumilast | Tiotropium + placebo |
| | (n = 466) | (n = 467) | (n = 371) | (n = 372) |
| Age (years)* | 65(9) | 65(9) | 64(9) | 64(9) |
| Men | 319(68%) | 299(64%) | 262(71%) | 267(72%) |
| Cigarette Pack-year*† | 43(22) | 43(22) | 43(22) | 42(22) |
| Smoking Status* | | | | |
| Current smoker | 184(39%) | 184(39%) | 147(40%) | 146(39%) |
| Former Smoker | 282(61%) | 283(61%) | 224(60%) | 226(61%) |
| Chronic cough and | 367(79%) | 362(78%) | 371(100%)‡ | 372(100%)‡ |
| sputum* | | | | |
| Prebronchodilator FEV₁ | 1.43(0.4) | 1.41(0.4) | 1.47(0.5) | 1.49(0.5) |
| (L)∫ | | | | |
| Postbronchodilator FEV₁ | 1.51(0.4) | 1.49(0.4) | 1.55(0.4) | 1.56(0.4) |
| (L)∫ | | | | |
| Prebronchodilator FEV₁ (% | 51.9(9.6) | 52.4(9.8) | 53.3(11.7) | 53.4(11.6) |
| of predicted)∫ | | | | |
| Postbronchodilator FEV₁ | 54.7(9.1) | 55.3(9.2) | 56.0(11.6) | 56.2(11.6) |
| (% of predicted)f | | | | |
| Postbronchodilator | 49.8(9.4) | 50.0(9.7) | 52.7(10.3) | 51.6(9.9) |
| FEV₁/FVC (%)∫ | | | | |
| Use of as-needed relievers¶ | 1.4(0-17.1) | 1.7(0-28.7) | 4.7(0-20.0) | 4.6(1.0-36.3) |
| (median, range) | | | | |
| COPD severity FEV₁*∥** | | | | |
| Moderate | 303(65%) | 324(69%) | 235(63%) | 240(65%) |
| Severe | 162(35%) | 141(30%) | 125(34%) | 119(32%) |

Data are mean (SD) or number (%) unless otherwise indicated.
FEV₁ = forced expiratory volume in 1 s.
FVC = forced vital capacity.
COPD = chronic obstructive pulmonary disease.
*Measurements were taken at the beginning of the run-in period.
†1 pack-year = 20 cigarettes per day for 1 year.
‡Assumed from study inclusion criteria.
∫Measurements were taken at baseline.
¶Puffs per day in salmeterol plus roflumilast trial; puffs per week in tiotropium plus roflumilast trial.
∥Based on the criteria of the Global initiative for chronic Obstructive Lung Disease
**Percentages do not add up to 100% because patients with mild or very severe COPD are not shown.

The study populations in the two trials did not differ. Most participants were elderly individuals, men, former smokers (more than 60%) with considerable previous tobacco consumption, and had moderate-to-severe airflow limitation (table above). As expected, the use of shortacting β2 agonists at baseline was higher in the tiotropium plus roflumilast trial than in the salmeterol plus roflumilast trial. Adherence to treatment was similar in all groups: the mean compliance was between 94% and 97%.

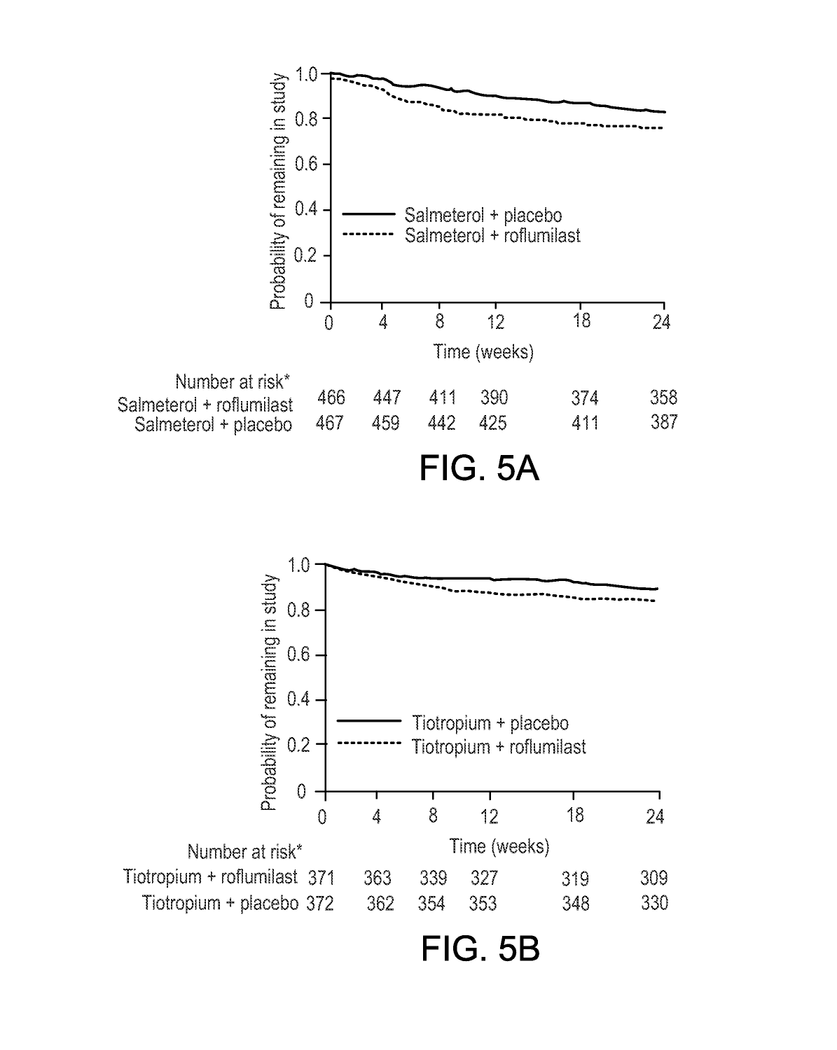

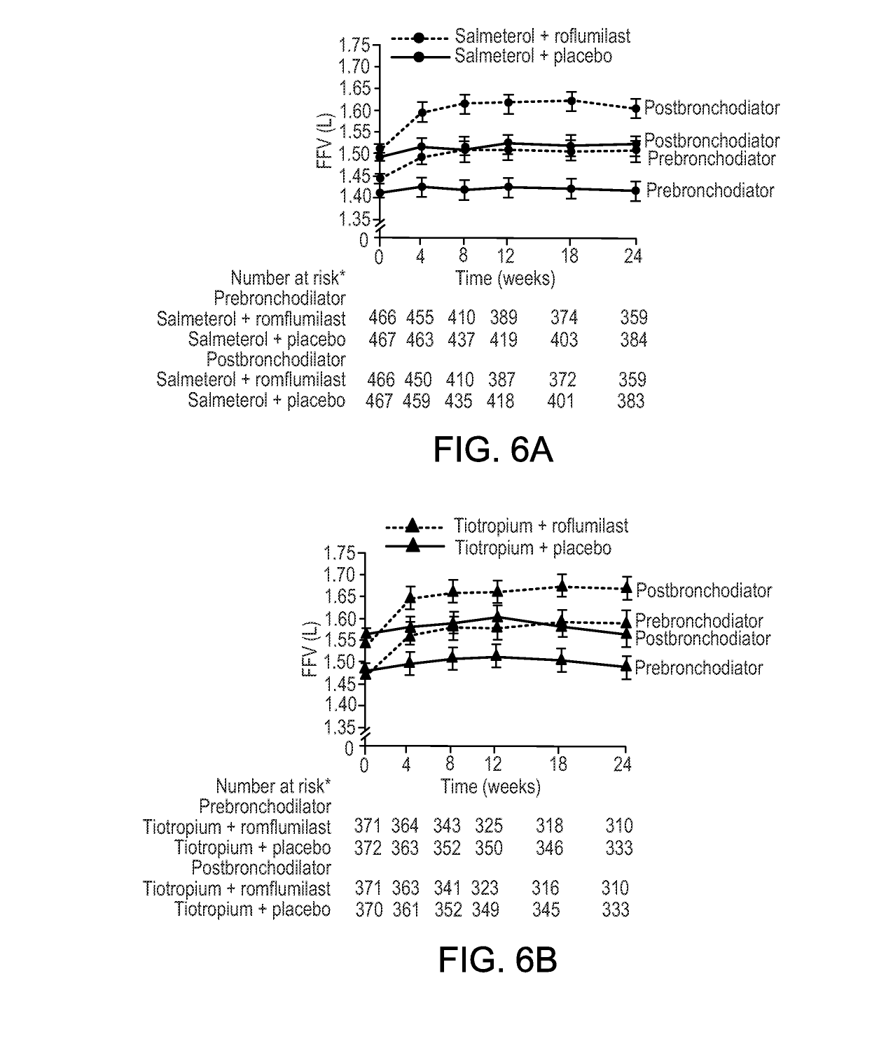

In both trials, the probability of treatment discontinuation was greater in patients treated with roflumilast (FIGS. 5A and 5B). The prebronchodilator FEV₁ increased significantly in patients in the roflumilast groups in both studies (FIGS. 6A and 6B). Similar improvements were noted in postbronchodilator FEV₁ and in prebronchodilator and postbronchodilator FVC (Table 5). The prebronchodilator changes in FEV₁ were similar in patients with different characteristics (eg, disease severity, sex, rescue use of shortacting bronchodilators, and current smoking status. The sensitivity analyses confirmed the robustness of the results for FEV₁ with respect to the effect of differential dropouts and missing data (data not shown)

Roflumilast had a variable effect on symptomatic outcomes such as respiratory symptoms, use of rescue medications, and exacerbations in both trials (Table 5). In general, the beneficial effect of roflumilast on some patient-reported outcomes (eg, TDI, SOBQ, use of rescue medication) was more pronounced in the tiotropium plus roflumilast trial than in the salmeterol plus roflumilast trial.

TABLE 5
Effect of treatment on primary and secondary functional and clinical outcomes in salmeterol + roflumilast (M2-127) and
tiotropium + roflumilast (M2-128) trials.

| | | | | M2-128 | | |
|---|---|---|---|---|---|---|
| | M2-127 | | | | | Tiotropium + |
| | Salmeterol + | Salmeterol + | Salmeterol + roflumilast | Tiotropium + | Tiotropium + | roflumilast vs |
| | roflumilast | placebo | vs Salmeterol + placebo | roflumilast | placebo | Tiotropium + placebo |
| Lung Function* | | | | | | |
| Change in | 39 (9); n = 456 | −10 (9); | Difference 49 | 65 (12); n = 365 | −16 (12); n = 364 | Difference 80 |
| prebronchodillator | | n = 463 | (27 to 71); p < 0.0001 | | | (51 to 110); p < 0.0001 |
| FEV₁ (mL) | | | | | | |
| Change in | 68 (9); n = 452 | 8 (9); n = 460 | Difference 60 | 74 (12); n = 364 | −7 (11); n = 363 | Difference 81 |
| postbronchodillator | | | (38 to 82); p < 0.0001 | | | (51 to 110); p < 0.0001 |
| FEV₁ (mL) | | | | | | |
| Change in | 32 (15); n = 456 | −14(14); | Difference 47 | 54 (20); n = 365 | −41 (19); n = 364 | Difference 95 |
| prebronchodillator | | n = 463 | (10 to 84); p = 0.0128 | | | (47 to 143); p = 0.0001 |
| FVC (mL) | | | | | | |
| Change in | 67 (15); n = 452 | 10 (15); | Difference 58 | 27 (23); n = 364 | −74 (22); n = 363 | Difference 101 |
| postbronchodillator | | n = 460 | (20 to 95); p = 0.0028 | | | (45 to 156); p = 0.0004 |
| FVC (mL) | | | | | | |
| Exacerbations† | | | | | | |
| Mild, moderate, or | 1.9 (1.5 to | 2.4 (1.9 to | RR 0.79 (0.58 to | 1.8 (1.3 to 2.5); | 2.2 (1.7 to 2.9); | RR 0.84 (0.57 to 1.23); |
| severe (mean rate, | 2.5); n = 131 | 3.1); n = 159 | 1.08); p = 0.1408 | n = 82 | n = 112 | p = 0.3573 |
| per patient per year | | | | | | |
| [95& CI]) | | | | | | |
| Median time to | 83.0 (41.0 to | 71.0 (33.0 to | HR 0.6 (0.4 to 0.9); | 80.5 (49.0 to | 74.5 (35.0 to | HR 0.8 (0.5 to 1.1); |
| first exacerbation | 102.0) | 109.0) | P = 0.0067 | 124.0) | 123.0) | p = 0.1959 |
| (moderate or severe, | | | | | | |
| days [IQR]) | | | | | | |
| Median time to | 53.0 (10.0 to | 47.0 (17.0 to | HR 0.9 (0.7 to 1.1); | 50.0 (15.0 to | 37.0 (13.0 to | HR 0.7 (0.5 to 1.0); |
| first exacerbation | 85.0) | 96.0) | p = 0.2707 | 98.0) | 88.0) | p = 0.0264 |
| (mild, moderate, or | | | | | | |
| severe events, | | | | | | |
| days [IQR]) | | | | | | |
| Proportion of patients | 51 (11%) | 83 (18%) | RiR 0.60 (0.43 to | 42 (11%) | 58 (16%) | RiR 0.73 (0.51 to 1.05); |
| with an exacerbation | | | 0.82); p = 0.0015 | | | p = 0.0867 |
| (moderate or severe) | | | | | | |
| Proportion of patients | 131 (28%) | 159 (34%) | RiR 0.82 (0.68 to | 82 (22%) | 112 (30%) | RiR 0.75 (0.59 to 0.95); |
| with an exacerbation | | | 0.99); p = 0.0419 | | | p = 0.0169 |
| (mild, moderate or | | | | | | |
| severe) | | | | | | |
| Further pre-specified | | | | | | |
| secondary analyses | | | | | | |
| TDI focal score* | 1.2 (0.1); | 1.1 (0.1); | Difference 0.1 (−0.2 to | 1.4 (0.1); n = 364 | 0.9 (0.1); n = 364 | Difference 0.4 |
| | n = 454 | n = 460 | 0.4); p = 0.4654 | | | (0.1 to 0.7); |
| | | | | | | p = 0.0032 |
| Change in SOBQ* | −0.6 (0.7); | −1.1 (0.7); | Difference 0.5 (−1.2 to | −3.4 (0.7); | −0.7 (0.7); | Differnece −2.6 |
| | n = 454 | n = 461 | 2.2); p = 0.5457 | n = 359 | n = 359 | (−4.5 to −0.8); |
| | | | | | | p = 0.0051 |
| Change from baseline | −0.01 (0.08); | 0.08 (0.08); | Difference −0.09 (−0.28 | −1.56 (0.11); | −1.05 (0.11); | Difference −0.51 |
| in rescue medication | n = 437 | n = 442 | to 0.11); p = 0.3689 | n = 364 | n = 365 | (−0.80 to −0.23); |
| (puffs per day)* | | | | | | p = 0.0004 |

Data are mean (SE), difference (95% CI), or point estimate (95% CI), unless otherwise indicated,
n = number of patients with data available (or, for exacerbations, number of patients with at least one exacerbation).
FEV₁ = forced expiratory volume in 1s. FVC = forced vital capacity.
RR = rate ratio.
HR = hazard ratio.
RiR = risk ratio,
TDI = transition dyspnoea index.
SOBQ = Shortness Of Breath Questionnaire.
*Least squares mean (SE).
‡Estimated exacerbation rates were based on a Poisson regression model. HRs were based on a Cox proportional hazards model.
RiRs were based on a log binomial regression model. Models included the treatment, age, sex, smoking status, county pool, and baseline postbronchodillator FEV₁ (only for the Poisson regression model).

In the salmeterol plus roflumilast trial, 294 (63%) patients assigned to salmeterol plus roflumilast reported 671 adverse events and 276 (59%) assigned to salmeterol plus placebo reported 598 adverse events. In the tiotropium plus roflumilast trial, 172 (46%) patients in the tiotropium plus roflumilast group reported 373 adverse events and 150 (41%) in the tiotropium plus placebo group reported 287 adverse events. Most roflumilast-associated events affected the gastrointestinal and respiratory tracts.

The most frequently reported adverse event in both studies was COPD related (Table 6). The number of patients with adverse events that were judged by the investigator to be related to treatment was 83 (18%) with salmeterol and roflumilast, 14 (3%) with salmeterol and placebo, 45 (12%) with tiotropium and roflumilast, and 6 (2%) with tiotropium and placebo. Diarrhea, nausea, and weight loss were the most common treatment-related adverse events, with no major difference between the two studies. Compared with placebo, roflumilast was associated with increased withdrawal from the study. This increase was significant in the salmeterol plus roflumilast trial (p=0.0019) but not in the tiotropium plus roflumilast trial (p=0.0864; FIG. 5).

TABLE 6
Adverse events occurring in at least 2% of patients in one of the treatment groups in the salmeterol plus
roflumilast (M2-127) and tiotropium plus roflumilast (M2-128) trials.

| | M2-127* | | | M2-128 | | |
|---|---|---|---|---|---|---|
| | Salmeterol + | Salmeterol + | Salmeterol + roflumilast | Tiotropium + | Tiotropium + | Tiotropium + roflumilast |
| | roflumilast | Placebo | vs salmeterol + placebo | roflumilast | placebo | vs tiotropium + placebo |
| | (n = 466) | (n = 467)† | (difference, 95% CI) | (n = 374)‡ | (n = 369)‡ | (difference, 95% CI) |
| COPD | 74 (16%) | 111 (24%) | −7.89 (−13.2 to 2.58) | 58 (16%) | 67 (18%) | −2.65% (−8.30 to −3.00) |
| Weight loss | 40 (9%) | 5 (1%) | 7.51% (4.59 to 10.44) | 21 (6%) | 2 (<1%) | 5.07% (2.35 to 7.79) |
| Diarrhoea | 38 (8%) | 16 (3%) | 4.73% (1.53 to 7.93) | 33 (9%) | >2 (<1%) | 8.28% (5.04 to 11.52) |
| Nasopharyngitis | 33 (7%) | 35 (7%) | −0.41% (−3.96 to 3.14) | 21 (6%) | 20 (5%) | 0.19% (−3.36 to 3.75) |
| Nausea | 25 (5%) | 1 (<1%) | 5.15% (2.85 to 7.45) | 11 (3%) | >4 (1%) | 1.86% (−0.42 to 4.14) |
| Headache | 14 (3%) | 5 (1%) | 1.93% (−0.09 to 3.96) | 8 (2%) | 0 | 2.14% (0.40 to 3.87) |
| Back pain | 13 (3%) | 9 (2%) | 0.86% (−1.30 to 3.02) | 7 (2%) | 5 (1%) | 0.52% (−1.56 to 2.60) |
| Bronchitis | 11 (2%) | ‘15 (3%) | −0.85% (−3.18 to 1.47) | 6 (2%) | 10 (3%) | −1.11% (−3.46 to 1.25) |
| Tremor | 10 (2%) | 2 (<1%) | 1.72% (0.06 to 3.37) | 0 | 2 (<1%) | −0.54% (−1.56 to 0.48) |
| Decreased | 10 (2%) | 1 (<1%) | 1.93% (0.34 to 3.53) | 3 (<1%) | 0 | ‘0.80% (−0.37 to 1.98) |
| appetite | | | | | | |
| Insomnia | 10 (2%) | 1 (<1%) | 1.93% (0.34 to 3.53) | 6 (2%) | 1 (<1%) | 1.33% (−0.32 to 2.98) |
| Upper | 9 (2%) | 19 (4%) | −2.14% (−4.54 to 0.26) | 4 (1%) | 2 (<1%) | 0.53% (−1.03 to 2.08) |
| respiratory tract | | | | | | |
| infection | | | | | | |
| Influenza | 9 (2%) | 11 (2%) | −0.42% (−2.50 to 1.65) | 3 (<1%) | 0 | 0.80% (−0.37 to 1.98) |
| Dyspnoea | 2 (<1%) | 14 (3%) | −2.57% (−4.44 to 0.70) | 3 (<1%) | 5 (1%) | −0.55% (−2.31 to 1.20) |

Data are number (%), unless otherwise indicated.
Adverse events are reported independently from investigator causality assessment. Patients might have had more than one adverse event.
COPD = chronic obstructive pulmonary disease.
*Incidence of adverse events in descending order.
†Three patients assigned to placebo were given roflumilast; 371 patients in tiotropium + roflumilast group and 372 in the tiotropium + placebo group were included in the efficacy analysis in study M2-128.

In both trials, similar gradual reductions were noted in mean body-weight in the roflumilast groups during the 24 weeks of treatment (salmeterol plus roflumilast trial −2.0 kg; tiotropium plus roflumilast trial −1.8 kg), whereas there was little change in the salmeterol or tiotropium plus placebo groups (salmeterol plus roflumilast trial +0.2 kg; tiotropium plus roflumilast trial +0.3 kg, Weight loss was similar in the two trials and was not significantly different between patients in different BMI categories. In the salmeterol plus roflumilast trial only, weight loss associated with roflumilast was greater in patients with gastrointestinal adverse events or headache, or both.

In patients with moderate-to-severe COPD treated with salmeterol or tiotropium, roflumilast improves lung function and some clinically relevant symptomatic outcomes. These results confirm the conclusions drawn from the findings of previous randomised clinical trials in which roflumilast was efficacious in patients with severe COPD who were not regularly treated with longacting bronchodilators. Additionally, the present results show that roflumilast maintains its clinical efficacy in patients with moderate-to-severe COPD who are already treated with longacting bronchodilators. However, these beneficial effects are also associated with some adverse effects of roflumilast. The improvement in prebronchodilator and postbronchodilator FEV₁ suggests that the beneficial effect of roflumilast on lung function is additive to that achieved with bronchodilators, an effect that is probably not primarily due to smooth muscle relaxation but to other mechanisms. Roflumilast has no direct effect on smooth muscle in most animal models, and, like other highly selective PDE4 inhibitors, has no appreciable acute bronchodilator effect in people. Also, roflumilast specifically inhibits PDE4, which is mainly expressed in inflammatory cells, and has no appreciable inhibitory effect on PDE3 at the doses administered.

The improvement in lung function obtained with the same dose used in the present studies is associated with a reduction in numbers of sputum neutrophils and eosinophils in patients with COPD. With consideration of all of the above, it is postulated that suppression of inflammation is likely to be the mechanism of the improvement in lung function induced by roflumilast in these studies. However, no effect of roflumilast was noted on concentrations of C-reactive protein or number of circulating leucocytes, another potential biomarker of systemic inflammation. Thus, additional studies are needed to investigate the mechanism of improvement in lung function provided by roflumilast in patients given longacting bronchodilators.

The additive effect of roflumilast on lung function is small but occurs in patients who are already being treated with effective, longacting bronchodilators and who have been screened for limited acute bronchodilator reversibility, but who are not taking inhaled corticosteroids. The improvement in lung function induced by roflumilast in patients with COPD concomitantly treated with salmeterol, noted in the present study, is similar to the improvement in lung function induced by inhaled corticosteroids in patients with COPD of similar severity and functional characteristics who were treated with salmeterol. Whether roflumilast would maintain this additive effect in patients concomitantly treated with longacting bronchodilators and glucocorticosteroids remains to be established.

The inclusion criteria for the tiotropium plus roflumilast trial led to the recruitment of more symptomatic patients with a higher use of as-needed medications than in the salmeterol plus roflumilast trial. The results from a post-hoc analysis of a previous study suggested that these characteristics might increase the chance of detecting an effect of roflumilast on patient-reported outcomes such as dyspnea and use of as-needed medications, and thus might explain the better efficacy of roflumilast that was noted in some patient-reported outcomes in tiotropium-treated patients than in salmeterol-treated patients. However, the designs of the present studies do not allow an indirect comparison of efficacy and safety between the salmeterol plus roflumilast and tiotropium plus roflumilast combinations. It should be noted that the current studies were powered to detect improvement in lung function, and the 6-month treatment duration of these trials was too short to allow reliable detection of an effect on some patient-reported outcomes, such as exacerbations. Additionally, the rate of exacerbations per year recorded during the study was low, probably because patients had COPD that was moderate to severe rather than severe to very severe, and because they were already treated with longacting bronchodilators that have been previously shown to reduce exacerbations.

Nevertheless, roflumilast did reduce some of the measures of exacerbation, particularly in patients treated with tiotropium, a result that must be interpreted cautiously because of the study design. Further studies are needed to investigate whether roflumilast has an additive effect on exacerbations when combined with longacting bronchodilators or used with a combination of inhaled bronchodilators and glucocorticosteroids. Although there are limitations and the variable effects on patient-reported outcomes, the consistent efficacy of roflumilast in terms of lung function lends support to the potential benefits of this treatment for patients with COPD that is moderate to severe who are already being treated with longacting bronchodilators.

PDE4 inhibitors have a well described adverse event profile. In the two trials reported here, the prevalence of drug-related adverse events, including weight loss, was similar to that reported in patients irregularly taking long-acting bronchodilators in other 12-month studies. Weight loss was greater in patients treated with roflumilast who had gastrointestinal adverse events or headache, or both, than in individuals who did not, suggesting that it might be causally related to these adverse events.

The use of an oral, once-daily anti-inflammatory agent instead of inhaled corticosteroids as concomitant therapy to longacting bronchodilators might have advantages and disadvantages. Important advantages associated with roflumilast might be increased compliance with oral once-daily administration, particularly in addition to once-daily tiotropium, which is not available in combination with steroids, and no demonstrable increased risk of pneumonia. By contrast, the adverse events associated with roflumilast constitute a disadvantage that could force some patients to discontinue this drug. The risks and benefits of the addition of roflumilast should be compared with a combination of bronchodilators or inhaled corticosteroids, or both, in large, well designed studies.

The adverse effects of roflumilast resemble some of those of theophylline, a drug with a weak and non-specific inhibitory effect on various phosphodiesterases and other pharmacological effects (eg, adenosine receptor antagonism). The pronounced differences in molecular structure and pharmacology between roflumilast and theophylline suggest that the adverse effects might result from different mechanisms of action.

Roflumilast improves lung function in patients with moderate-to-severe COPD who are already being treated with longacting bronchodilators (β2 agonists or anticholinergic drugs), although with expected class-specific adverse events.

What is claimed is:

1. A method of treatment of chronic obstructive pulmonary disease associated with chronic bronchitis in a patient at risk of exacerbations, comprising:
administering to a patient with a history of chronic obstructive pulmonary disease associated with chronic bronchitis and at risk of exacerbations, a maintenance dose of 500 micrograms per day of roflumilast.

2. A method of treatment of chronic obstructive pulmonary disease, comprising:
identifying a patient with a history of chronic obstructive pulmonary disease associated with chronic bronchitis; and
administering a maintenance dose to said patient of 500 micrograms per day of roflumilast.

3. The method of claim 2, wherein said patient has a non-reversible airway obstruction (post-bronchodilator FEV₁/FVC ratio of less than or equal to 70%).

4. The method of claim 3, wherein said patient further has an FEV₁ of less than or equal to 50% of the predicted FEV₁.

5. The method of claim 4, wherein said patient has had at least one exacerbation during the 12 months prior to administration of roflumilast.

6. A method of increasing pre-bronchodilator FEV₁ or post-bronchodilator FEV₁ in a patient with a history of chronic obstructive pulmonary disease associated with chronic bronchitis, comprising:
administering a maintenance dose to a patient with a history of chronic obstructive pulmonary disease associated with chronic bronchitis of 500 micrograms per day of roflumilast.

7. The method of claim 6 further comprising,
assessing a first pre-bronchodilator FEV₁ or post-bronchodilator FEV₁ in a patient with a history of chronic obstructive pulmonary disease associated with chronic bronchitis; and after
administering a maintenance dose to said patient of 500 micrograms per day of roflumilast, subsequently assessing a second pre-bronchodilator FEV₁ or post-bronchodilator FEV₁, respectively, in said patient.

8. The method of claim 6, wherein said patient has a non-reversible airway obstruction (post-bronchodilator FEV₁/FVC ratio of less than or equal to 70%).

9. The method of claim 8, wherein said patient further has an FEV₁ of less than or equal to 50% of the predicted FEV₁.

10. The method of claim 9, wherein said patient has had at least one exacerbation during the 12 months prior to administration of roflumilast.

11. A method of increasing pre-bronchodilator FVC or post-bronchodilator FVC in a patient with a history of chronic obstructive pulmonary disease associated with chronic bronchitis, comprising:
administering a maintenance dose to a patient with a history of chronic obstructive pulmonary disease associated with chronic bronchitis of 500 micrograms per day of roflumilast.

12. The method of claim 11, further comprising,
assessing a first pre-bronchodilator FVC or post-bronchodilator FVC in a patient with a history of chronic obstructive pulmonary disease associated with chronic bronchitis; and after
administering a maintenance dose to said patient of 500 micrograms per day of roflumilast, subsequently assessing a second pre-bronchodilator FVC or post-bronchodilator FVC, respectively, in said patient.

13. The method of claim 11, wherein said patient has a non-reversible airway obstruction (post-bronchodilator FEV₁/FVC ratio of less than or equal to 70%).

14. The method of claim 13, wherein said patient further has an FEV₁ of less than or equal to 50% of the predicted FEV₁.

15. The method of claim 14, wherein said patient has had at least one exacerbation during the 12 months prior to administration of roflumilast.

16. A method of reducing the rate of exacerbations in a patient with a history of chronic obstructive pulmonary disease associated with chronic bronchitis, comprising:
administering a maintenance dose to a patient with a history of chronic obstructive pulmonary disease associated with chronic bronchitis of 500 micrograms per day of roflumilast.

17. The method of claim 16, further comprising,
assessing a first rate of exacerbations in a patient with a history of chronic obstructive pulmonary disease associated with chronic bronchitis; and after
administering a maintenance dose to said patient of 500 micrograms per day of roflumilast, subsequently assessing a second rate of exacerbations in said patient.

18. The method of claim 16, wherein said patient has a non-reversible airway obstruction (post-bronchodilator FEV₁/FVC ratio of less than or equal to 70%).

19. The method of claim 18, wherein said patient further has an FEV₁ of less than or equal to 50% of the predicted FEV₁.

20. The method of claim 19, wherein said patient has had at least one exacerbation during the 12 months prior to administration of roflumilast.

21. The method of claim 16 wherein said exacerbations are moderate.

22. The method of claim 16 wherein said exacerbations are severe.

23. The method of claim 16, wherein said exacerbations include both moderate and severe exacerbations.

24. The method of claim 1, wherein said dose of roflumilast is administered once per day.

25. The method of claim 24, wherein said dose of roflumilast is administered orally.

26. The method of claim 25, wherein said dose of roflumilast is administered as a tablet.

27. The method of claim 26, wherein said tablet is D-shaped.

28. The method of claim 27, wherein said tablet is film-coated.

29. The method of claim 1, wherein said patient has experienced a history of chronic obstructive pulmonary disease for at least 12 months prior to administration of said roflumilast.

30. The method of claim 5, wherein said patient has experienced a history of chronic obstructive pulmonary disease for at least 12 months prior to administration of said roflumilast.

31. The method of claim 29, wherein said patient has been treated with a long-acting bronchodilator prior to administration of said roflumilast.

32. The method of claim 31, wherein said treatment with said long-acting bronchodilator is continued concurrently with maintenance administration of roflumilast.

33. The method of claim 31, wherein said long-acting bronchodilator is selected from the group consisting of tiotropium bromide, tiotropium bromide monohydrate, salmeterol, formoterol, aformoterol and combinations thereof.

34. The method of claim 31, wherein said long-acting bronchodilator is selected from the group consisting of salmeterol, tiotropium bromide, tiotropium bromide monohydrate and combinations thereof.

35. The method of claim 2, wherein said patient is administered a dose of less than 500 micrograms per day of roflumilast if said patient is being treated with a CYP 1A2 or dual CYP 3A4/1A2 inhibitor.

36. The method of claim 35, wherein said CYP 1A2 or dual CYP 3A4/1A2 inhibitor is selected from the group consisting of fluvoxamine, enoxacine and cimetidine and mixtures thereof.

37. The method of claim 2, wherein said patient is administered a dose of greater than 500 micrograms per day of roflumilast if said patient is being treated with a cytochrome P450 enzyme inducer.

38. The method of claim 37, wherein said cytochrome P450 enzyme is selected from the group consisting of rifampicin, phenobarbital, carbamazepine and phenyloin and mixtures thereof.

39. The method of claim 2, wherein said treatment includes concomitant administration of a long-acting beta-2 agonist.

40. The method of claim 5, wherein said treatment includes concomitant administration of a long-acting beta-2 agonist.

41. The method of claim 2, wherein said treatment excludes concomitant administration of an inhaled corticosteroid.

42. The method of claim 5, wherein said treatment excludes concomitant administration of an inhaled corticosteroid.

43. The method of claim 2, wherein said treatment includes concomitant administration of an inhaled corticosteroid.

44. The method of claim 5, wherein said treatment includes concomitant administration of an inhaled corticosteroid.

45. A method to reduce the risk of chronic obstructive pulmonary disease exacerbations in patients with severe chronic obstructive pulmonary disease associated with chronic bronchitis and a history of exacerbations, comprising:
administering to a patient with severe chronic obstructive pulmonary disease associated with chronic bronchitis and a history of exacerbations,
a dose of 500 micrograms per day of roflumilast.

46. The method of claim 45, wherein said dose is administered in tablet form.

47. The method of claim 45, wherein said patient has a non-reversible airway obstruction (post-bronchodilator FEV₁/FVC ratio of less than or equal to 70%).

48. The method of claim 47, wherein said patient further has an FEV₁ of less than or equal to 50% of the predicted FEV₁.

49. The method of claim 48, wherein said patient has had at least one exacerbation during the 12 months prior to administration of roflumilast.
